SEQUENCE LISTING (59 sequences)

SEQ ID NO: 1 - protein, 11 aa, synthetic construct
ASHLRKLRKRL

SEQ ID NO: 2 - protein, 8 aa, synthetic construct
  SITE 1: note = the N-terminus is acetylated
  SITE 2: note = 6-aminohexanoic acid
  SITE 6: note = hydroxyproline
  SITE 8: note = the C-terminus is amidated
KXALAPYI

SEQ ID NO: 3 - protein, 8 aa, synthetic construct
  SITE 1: note = the N-terminus is acetylated
  SITE 8: note = the C-terminus is amidated
  SITE 1: note = epsilon-azido-lysine
  SITE 2: note = 6-aminohexanoic acid
  SITE 6: note = hydroxyproline
KXALAPYI

SEQ ID NO: 4 - protein, 8 aa, synthetic construct
  SITE 1: note = the N-terminus is acetylated
  SITE 8: note = the C-terminus is amidated
  SITE 1: note = propargylglycine
  SITE 2: note = 6-aminohexanoic acid
  SITE 6: note = hydroxyproline
GXALAPYI

SEQ ID NO: 5 - protein, 8 aa, synthetic construct
  SITE 1: note = the N-terminus is acetylated
  SITE 8: note = the C-terminus is amidated
  SITE 2: note = 6-aminohexanoic acid
  SITE 6: note = hydroxyproline
CXALAPYI

SEQ ID NO: 6 - protein, 14 aa, synthetic construct
  SITE 1: note = the N-terminus is acetylated
  SITE 14: note = the C-terminus is amidated
  SITE 2: note = 6-aminohexanoic acid
KXASHLRKLRKRLL

SEQ ID NO: 7 - protein, 14 aa, synthetic construct
  SITE 1: note = the N-terminus is acetylated
  SITE 14: note = the C-terminus is amidated
  SITE 1: note = epsilon-azido-lysine
  SITE 2: note = 6-aminohexanoic acid
KXASHLRKLRKRLL

SEQ ID NO: 8 - protein, 14 aa, synthetic construct
  SITE 1: note = the N-terminus is acetylated
  SITE 14: note = the C-terminus is amidated
  SITE 1: note = propargylglycine
  SITE 2: note = 6-aminohexanoic acid
GXASHLRKLRKRLL

SEQ ID NO: 9 - protein, 14 aa, synthetic construct
  SITE 1: note = the N-terminus is acetylated
  SITE 14: note = the C-terminus is amidated
  SITE 2: note = 6-aminohexanoic acid
CXASHLRKLRKRLL

SEQ ID NO: 10 - protein, 7 aa, synthetic construct
  SITE 1: note = the N-terminus is acetylated
  SITE 7: note = the C-terminus is amidated
  SITE 2: note = 6-aminohexanoic acid
KXVSKRR

SEQ ID NO: 11 - protein, 7 aa, synthetic construct
  SITE 1: note = the N-terminus is acetylated
  SITE 7: note = the C-terminus is amidated
  SITE 1: note = epsilon-azido-lysine
  SITE 2: note = 6-aminohexanoic acid
KXVSKRR

SEQ ID NO: 12 - protein, 7 aa, synthetic construct
  SITE 1: note = the N-terminus is acetylated
  SITE 7: note = the C-terminus is amidated
  SITE 1: note = propargylglycine
  SITE 2: note = 6-aminohexanoic acid
GXVSKRR

SEQ ID NO: 13 - protein, 7 aa, synthetic construct
  SITE 1: note = the N-terminus is acetylated
  SITE 7: note = the C-terminus is amidated
  SITE 2: note = 6-aminohexanoic acid
CXVSKRR

SEQ ID NO: 14 - protein, 7 aa, synthetic construct
  SITE 7: note = the C-terminus is amidated
  SITE 1: note = conjugated to 3,5-diamino-6-chloro-N-(diaminomethyl idene)pyrazine-2-carboxamide
  SITE 1: note = 6-aminohexanoic acid
  SITE 5: note = hydroxyproline
XALAPYI

SEQ ID NO: 15 - protein, 8 aa, synthetic construct
  SITE 1: note = the N-terminus is acetylated
  SITE 8: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
  SITE 6: note = hydroxyproline
KXALAPYI

SEQ ID NO: 16 - protein, 8 aa, synthetic construct
  SITE 1: note = the N-terminus is acetylated
  SITE 8: note = the C-terminus is amidated
  SITE 1: note = conjugated to 3,5-diamino-6-chloro-N-(diaminomethyl idene)pyrazine-2-carboxamide
  SITE 1: note = epsilon-azido-lysine
  SITE 2: note = 6-aminohexanoic acid
  SITE 6: note = hydroxyproline
KXALAPYI

SEQ ID NO: 17 - protein, 8 aa, synthetic construct
  SITE 1: note = the N-terminus is acetylated
  SITE 8: note = the C-terminus is amidated
  SITE 1: note = conjugated to 3,5-diamino-6-chloro-N-(diaminomethyl idene)pyrazine-2-carboxamide
  SITE 1: note = propargylglycine
  SITE 2: note = 6-aminohexanoic acid
  SITE 6: note = hydroxyproline
GXALAPYI

SEQ ID NO: 18 - protein, 8 aa, synthetic construct
  SITE 1: note = the N-terminus is acetylated
  SITE 8: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
  SITE 6: note = hydroxyproline
CXALAPYI

SEQ ID NO: 19 - protein, 8 aa, synthetic construct
  SITE 1: note = the N-terminus is acetylated
  SITE 8: note = the C-terminus is amidated
  SITE 1: note = conjugated to 3,5-diamino-N-(N'-benzylcarbamimidoyl )-6-chloropyrazine-2-carboxamide
  SITE 1: note = epsilon-azido-lysine
  SITE 2: note = 6-aminohexanoic acid
  SITE 6: note = hydroxyproline
KXALAPYI

SEQ ID NO: 20 - protein, 8 aa, synthetic construct
  SITE 1: note = the N-terminus is acetylated
  SITE 8: note = the C-terminus is amidated
  SITE 1: note = conjugated to 3,5-diamino-N-(N'-benzylcarbamimidoyl )-6-chloropyrazine-2-carboxamide
  SITE 1: note = propargylglycine
  SITE 2: note = 6-aminohexanoic acid
  SITE 6: note = hydroxyproline
GXALAPYI

SEQ ID NO: 21 - protein, 8 aa, synthetic construct
  SITE 1: note = the N-terminus is acetylated
  SITE 8: note = the C-terminus is amidated
  SITE 1: note = conjugated to 3,5-diamino-N-(N'-benzylcarbamimidoyl )-6-chloropyrazine-2-carboxamide
  SITE 2: note = 6-aminohexanoic acid
  SITE 6: note = hydroxyproline
CXALAPYI

SEQ ID NO: 22 - protein, 13 aa, synthetic construct
  SITE 13: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
XASHLRKLRKRLL

SEQ ID NO: 23 - protein, 14 aa, synthetic construct
  SITE 1: note = the N-terminus is acetylated
  SITE 14: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
KXASHLRKLRKRLL

SEQ ID NO: 24 - protein, 14 aa, synthetic construct
  SITE 1: note = the N-terminus is acetylated
  SITE 14: note = the C-terminus is amidated
  SITE 1: note = conjugated to 3,5-diamino-6-chloro-N-(diaminomethyl idene)pyrazine-2-carboxamide
  SITE 1: note = epsilon-azido-lysine
  SITE 2: note = 6-aminohexanoic acid
KXASHLRKLRKRLL

SEQ ID NO: 25 - protein, 14 aa, synthetic construct
  SITE 1: note = the N-terminus is acetylated
  SITE 14: note = the C-terminus is amidated
  SITE 1: note = conjugated to 3,5-diamino-6-chloro-N-(diaminomethyl idene)pyrazine-2-carboxamide
  SITE 1: note = propargylglycine
  SITE 2: note = 6-aminohexanoic acid
GXASHLRKLRKRLL

SEQ ID NO: 26 - protein, 14 aa, synthetic construct
  SITE 1: note = the N-terminus is acetylated
  SITE 14: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
CXASHLRKLRKRLL

SEQ ID NO: 27 - protein, 7 aa, synthetic construct
  SITE 1: note = the N-terminus is acetylated
  SITE 7: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
CXVSKRR

SEQ ID NO: 28 - protein, 14 aa, synthetic construct
  SITE 1: note = the N-terminus is acetylated
  SITE 14: note = the C-terminus is amidated
  SITE 1: note = conjugated to 3,5-diamino-N-(N'-benzylcarbamimidoyl )-6-chloropyrazine-2-carboxamide
  SITE 1: note = propargylglycine
  SITE 2: note = 6-aminohexanoic acid
GXASHLRKLRKRLL

SEQ ID NO: 29 - protein, 14 aa, synthetic construct
  SITE 1: note = the N-terminus is acetylated
  SITE 14: note = the C-terminus is amidated
  SITE 1: note = conjugated to 3,5-diamino-N-(N'-benzylcarbamimidoyl )-6-chloropyrazine-2-carboxamide
  SITE 2: note = 6-aminohexanoic acid
CXASHLRKLRKRLL

SEQ ID NO: 30 - protein, 6 aa, synthetic construct
  SITE 1: note = the N-terminus is acetylated
  SITE 6: note = the C-terminus is amidated
  SITE 1: note = conjugated to 3,5-diamino-6-chloro-N-(diaminomethyl idene)pyrazine-2-carboxamide
  SITE 1: note = 6-aminohexanoic acid
XVSKRR

SEQ ID NO: 31 - protein, 7 aa, synthetic construct
  SITE 1: note = the N-terminus is acetylated
  SITE 7: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
KXVSKRR

SEQ ID NO: 32 - protein, 7 aa, synthetic construct
  SITE 1: note = the N-terminus is acetylated
  SITE 7: note = the C-terminus is amidated
  SITE 1: note = conjugated to 3,5-diamino-6-chloro-N-(diaminomethyl idene)pyrazine-2-carboxamide
  SITE 1: note = epsilon-azido-lysine
  SITE 2: note = 6-aminohexanoic acid
KXVSKRR

SEQ ID NO: 33 - protein, 7 aa, synthetic construct
  SITE 1: note = the N-terminus is acetylated
  SITE 7: note = the C-terminus is amidated
  SITE 1: note = conjugated to 3,5-diamino-6-chloro-N-(diaminomethyl idene)pyrazine-2-carboxamide
  SITE 1: note = propargylglycine
  SITE 2: note = 6-aminohexanoic acid
GXVSKRR

SEQ ID NO: 34 - protein, 7 aa, synthetic construct
  SITE 1: note = the N-terminus is acetylated
  SITE 7: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
CXVSKRR

SEQ ID NO: 35 - protein, 7 aa, synthetic construct
  SITE 1: note = the N-terminus is acetylated
  SITE 7: note = the C-terminus is amidated
  SITE 1: note = conjugated to 3,5-diamino-N-(N'-benzylcarbamimidoyl )-6-chloropyrazine-2-carboxamide
  SITE 1: note = epsilon-azido-lysine
  SITE 2: note = 6-aminohexanoic acid
KXVSKRR

SEQ ID NO: 36 - protein, 7 aa, synthetic construct
  SITE 1: note = the N-terminus is acetylated
  SITE 7: note = the C-terminus is amidated
  SITE 1: note = conjugated to 3,5-diamino-N-(N'-benzylcarbamimidoyl )-6-chloropyrazine-2-carboxamide
  SITE 1: note = propargylglycine
  SITE 2: note = 6-aminohexanoic acid
GXVSKRR

SEQ ID NO: 37 - protein, 7 aa, synthetic construct
  SITE 1: note = the N-terminus is acetylated
  SITE 7: note = the C-terminus is amidated
  SITE 1: note = conjugated to 3,5-diamino-N-(N'-benzylcarbamimidoyl )-6-chloropyrazine-2-carboxamide
  SITE 2: note = 6-aminohexanoic acid
CXVSKRR

SEQ ID NO: 38 - protein, 8 aa, synthetic construct
  SITE 1: note = the N-terminus is acetylated
  SITE 8: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
  SITE 6: note = hydroxyproline
KXALAPYI

SEQ ID NO: 39 - protein, 14 aa, synthetic construct
  SITE 1: note = the N-terminus is acetylated
  SITE 14: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
KXASHLRKLRKRLL

SEQ ID NO: 40 - protein, 7 aa, synthetic construct
  SITE 1: note = the N-terminus is acetylated
  SITE 7: note = the C-terminus is amidated
  SITE 1: note = conjugated to 3,5-diamino-6-chloro-N-(diaminomethyl idene)pyrazine-2-carboxamide
  SITE 2: note = 6-aminohexanoic acid
KXVSKRR

SEQ ID NO: 41 - protein, 8 aa, synthetic construct
  SITE 1: note = the N-terminus is acetylated
  SITE 8: note = the C-terminus is amidated
  SITE 1: note = conjugated to (E)-N-(((5-(1H-1,2,3-triazol-4-yl)pen tyl)amino)(amino)methylene)-3,5-diamino-6-chloropyrazine-2 -carboxamide
  SITE 1: note = epsilon-azido-lysine
  SITE 2: note = 6-aminohexanoic acid
  SITE 6: note = hydroxyproline
KXALAPYI

SEQ ID NO: 42 - protein, 14 aa, synthetic construct
  SITE 1: note = the N-terminus is acetylated
  SITE 14: note = the C-terminus is amidated
  SITE 1: note = conjugated to (E)-N-(((5-(1H-1,2,3-triazol-4-yl)pen tyl)amino)(amino)methylene)-3,5-diamino-6-chloropyrazine-2 -carboxamide
  SITE 1: note = epsilon-azido-lysine
  SITE 2: note = 6-aminohexanoic acid
KXASHLRKLRKRLL

SEQ ID NO: 43 - protein, 7 aa, synthetic construct
  SITE 1: note = the N-terminus is acetylated
  SITE 7: note = the C-terminus is amidated
  SITE 1: note = conjugated to (E)-N-(((5-(1H-1,2,3-triazol-4-yl)pen tyl)amino)(amino)methylene)-3,5-diamino-6-chloropyrazine-2 -carboxamide
  SITE 1: note = epsilon-azido-lysine
  SITE 2: note = 6-aminohexanoic acid
KXVSKRR

SEQ ID NO: 44 - protein, 8 aa, synthetic construct
  SITE 1: note = the N-terminus is acetylated
  SITE 8: note = the C-terminus is amidated
  SITE 1: note = conjugated to (E)-N-(((4-(1H-1,2,3-triazol-1-yl)but yl)amino)(amino)methylene)-3,5-diamino-6-chloropyrazine-2- carboxamide
  SITE 1: note = propargylglycine
  SITE 2: note = 6-aminohexanoic acid
  SITE 6: note = hydroxyproline
GXALAPYI

SEQ ID NO: 45 - protein, 14 aa, synthetic construct
  SITE 1: note = the N-terminus is acetylated
  SITE 14: note = the C-terminus is amidated
  SITE 1: note = conjugated to (E)-N-(((4-(1H-1,2,3-triazol-1-yl)but yl)amino)(amino)methylene)-3,5-diamino-6-chloropyrazine-2- carboxamide
  SITE 1: note = propargylglycine
  SITE 2: note = 6-aminohexanoic acid
GXASHLRKLRKRLL

SEQ ID NO: 46 - protein, 7 aa, synthetic construct
  SITE 1: note = the N-terminus is acetylated
  SITE 7: note = the C-terminus is amidated
  SITE 1: note = conjugated to (E)-N-(((4-(1H-1,2,3-triazol-1-yl)but yl)amino)(amino)methylene)-3,5-diamino-6-chloropyrazine-2- carboxamide
  SITE 1: note = 6-aminohexanoic acid
GXVSKRR

SEQ ID NO: 47 - protein, 8 aa, synthetic construct
  SITE 1: note = the N-terminus is acetylated
  SITE 8: note = the C-terminus is amidated
  SITE 1: note = conjugated to (E)-3,5-diamino-N-(amino((4-(3-mercap to-2,5-dioxopyrrolidin-1-yl)butyl)amino)methylene)-6-chlor opyrazine-2-carboxamide
  SITE 2: note = 6-aminohexanoic acid
  SITE 6: note = hydroxyproline
CXALAPYI

SEQ ID NO: 48 - protein, 14 aa, synthetic construct
  SITE 1: note = the N-terminus is acetylated
  SITE 14: note = the C-terminus is amidated
  SITE 1: note = conjugated to (E)-3,5-diamino-N-(amino((4-(3-mercap to-2,5-dioxopyrrolidin-1-yl)butyl)amino)methylene)-6-chlor opyrazine-2-carboxamide
  SITE 2: note = 6-aminohexanoic acid
CXASHLRKLRKRLL

SEQ ID NO: 49 - protein, 7 aa, synthetic construct
  SITE 1: note = the N-terminus is acetylated
  SITE 1: note = conjugated to (E)-3,5-diamino-N-(amino((4-(3-mercap to-2,5-dioxopyrrolidin-1-yl)butyl)amino)methylene)-6-chlor opyrazine-2-carboxamide
  SITE 2: note = 6-aminohexanoic acid
  SITE 7: note = the C-terminus is amidated
CXVSKRR

SEQ ID NO: 50 - protein, 13 aa, synthetic construct
  MOD_RES 2: note = aib
  SITE 13: note = the C-terminus is amidated
  SITE 1: note = conjugated to (E)-N-(((5-(1H-1,2,3-triazol-4-yl)pen tyl)amino)(amino)methylene)-3,5-diamino-6-chloropyrazine-2 -carboxamide
  SITE 1: note = the N-terminus is acetylated
KXSHLRKLRKRLL

SEQ ID NO: 51 - protein, 13 aa, synthetic construct
  SITE 1: note = conjugated to (E)-3,5-diamino-N-(amino((1-phenyl-5- (1H-1,2,3-triazol-4-yl)pentyl)amino)methylene)-6-chloropyr azine-2-carboxamide
  MOD_RES 2: note = aib
  SITE 13: note = the C-terminus is amidated
  SITE 1: note = the N-terminus is acetylated
KXSHLRKLRKRLL

SEQ ID NO: 52 - protein, 13 aa, synthetic construct
  SITE 1: note = conjugated to (E)-N-(((5-(1H-1,2,3-triazol-4-yl)pen tyl)amino)(amino)methylene)-3,5-diamino-6-chloropyrazine-2 -carboxamide
  SITE 2: note = D-Alanine
  SITE 13: note = the C-terminus is amidated
  SITE 1: note = the N-terminus is acetylated
KASHLRKLRKRLL

SEQ ID NO: 53 - protein, 13 aa, synthetic construct
  SITE 1: note = conjugated to 3,5-diamino-6-chloro-N-(diaminomethyl idene)pyrazine-2-carboxamide
  MOD_RES 2: note = aib
  SITE 13: note = the C-terminus is amidated
  SITE 1: note = the N-terminus is acetylated
KXSHLRKLRKRLL

SEQ ID NO: 54 - protein, 13 aa, synthetic construct
  SITE 1: note = conjugated to 3,5-diamino-N-(N'-benzylcarbamimidoyl )-6-chloropyrazine-2-carboxamide
  MOD_RES 2: note = aib
  SITE 13: note = the C-terminus is amidated
  SITE 1: note = the N-terminus is acetylated
KXSHLRKLRKRLL

SEQ ID NO: 55 - protein, 13 aa, synthetic construct
  SITE 1: note = conjugated to 3,5-diamino-6-chloro-N-(diaminomethyl idene)pyrazine-2-carboxamide
  SITE 2: note = D-Alanine
  SITE 13: note = the C-terminus is amidated
  SITE 1: note = the N-terminus is acetylated
KASHLRKLRKRLL

SEQ ID NO: 56 - protein, 13 aa, synthetic construct
  MOD_RES 2: note = aib
  SITE 13: note = the C-terminus is amidated
  SITE 1: note = the N-terminus is acetylated
KXSHLRKLRKRLL

SEQ ID NO: 57 - protein, 13 aa, synthetic construct
  SITE 2: note = D-Alanine
  SITE 13: note = the C-terminus is amidated
  SITE 1: note = the N-terminus is acetylated
KASHLRKLRKRLL

SEQ ID NO: 58 - protein, 11 aa, synthetic construct
SHLRKLRKRLL

SEQ ID NO: 59 - protein, 12 aa, synthetic construct
ASHLRKLRKRLL

(19) United States
(12) Patent Application Publication
(10) Pub. No.: US 2024/0374585 A1
(43) Pub. Date: Nov. 14, 2024

(54) EPITHELIAL SODIUM CHANNEL (ENAC) INHIBITOR CONJUGATES AND METHODS FOR USE THEREOF

(71) Applicant: Lung Therapeutics, Inc., Austin, TX (US)

(72) Inventor: Dale J. CHRISTENSEN, Austin, TX (US)

(21) Appl. No.: 18/577,028

(22) PCT Filed: Jul. 8, 2022
(86) PCT No.: PCT/US2022/073561

§ 371 Date: Jan. 5, 2024

Related U.S. Application Data
(60) Provisional application No. 63/243,629, filed on Sep. 13, 2021.
(60) Provisional application No. 63/219,488, filed on Jul. 8, 2021.

(51) Int. Cl. A61K 31/4965; A61K 9/00; A61K 47/64; A61P 11/00; C07K 7/06; C07K 7/08

(52) U.S. Cl. CPC A61K 31/4965; A61K 9/0073; A61K 47/64; A61P 11/00; C07K 7/06; C07K 7/08

(57) ABSTRACT

This disclosure relates to epithelial sodium channel (ENaC) inhibitory conjugates, compositions thereof, and method of use thereof. In particular the ENaC inhibitory conjugates of the present disclosure comprise peptides linked to Amiloride, and are useful for treating pulmonary diseases or disorder, including cystic fibrosis, chronic obstructive pulmonary disease, asthma, emphysema, primary ciliary dyskinesia, or pneumonia.

### CROSS-REFERENCE TO RELATED APPLICATIONS

This application claims the benefit of priority to U.S. Provisional Application No. 63/219,488, filed on Jul. 8, 2021, and U.S. Provisional Application No. 63/243,629, filed on Sep. 13, 2021, which are hereby incorporated by reference in their entireties.

### TECHNICAL FIELD

This disclosure relates to the fields of medicine, pharmacology, and chemistry. In particular, compounds, compositions, methods of treatment, and methods of synthesis relating to conjugate epithelial sodium channel (ENaC) inhibitors are disclosed.

### INCORPORATION OF THE SEQUENCE LISTING

The Sequence Listing associated with this application is provided in extensible Markup Language (XML) format in lieu of a paper copy and is hereby incorporated by reference into the specification. A computer readable format copy of the Sequence Listing (filename: LUTX_010_02WO_SeqList_ST26; date recorded: Jul. 7, 2022: file size: 142 KB) is submitted.

### BACKGROUND

Cystic fibrosis (CF) patients lack functional cystic fibrosis transmembrane conductance regulator (CFTR) Cl-channels, leading to chronic lung disease and digestive problems. Recently, it was shown that CFTR dysregulation in CF is associated with abnormal regulation of the ENaC, which leads to Na+ hyperabsorption. The combination of Na+ hyperabsorption and lack of Cl secretion is directly associated with dehydrated airway surface liquid (ASL) and thickened/dehydrated mucus that accumulates in the lung. ASL depletion due to dysfunctional CFTR/ENaC causes mucus to adhere to airway surfaces, preventing its clearance, allowing concentrated mucus plaques to accumulate until the airways become occluded and colonized by bacteria.

Epithelial sodium channel (ENaC) inhibitors have been explored for the therapeutic treatment of cystic fibrosis and other pulmonary diseases including primary ciliary dyskinesia. The ENaC inhibitors that have progressed into human clinical trials include small molecule inhibitors such as amiloride and benzamil along with other compounds such as VX-371 (Vertex Pharmaceuticals, also known as P1037 from Parion Sciences). Unfortunately for patients, the small molecule ENaC inhibitors have not demonstrated efficacy in human clinical trials due to rapid uptake into systemic circulation and toxic effects in the kidney, where inhibition of the renal ENaC results in concomitant inhibition of sodium potassium exchange pumps in the kidney that results in buildup of potassium in the blood (i.e., hyperkalemia) that prevents them from being dosed at sufficient concentrations to reach an efficacious lung dose.

More recently, peptide inhibitors of ENAC are being tested that are derived from the SPLUNC1 protein that is an endogenous inhibitor of ENAC that is expressed primarily in the lung. These SPLUNC-derived peptides are currently in clinical trials but have not yet reported results in trials designed to measure efficacy but have demonstrated that they are safe at high doses without appreciable systemic exposure.

In recent years, ENaC inhibition has been tested as an approach to aid in mucus rehydration in CF by retarding the hyperabsorption in CF. In the earliest trials, amiloride was delivered by nebulization to the lung of CF patients, but the trial failed to demonstrate improved lung function at doses that did not induce hyperkalemia. Additional clinical trials of small molecule ENaC inhibitors for the treatment of CF have failed because, like amiloride, these compounds are systemically absorbed and caused renal toxicity if dosed to levels that might be required for efficacy.

Thus, there is a need for efficacious ENaC inhibitors that are not systemically absorbed to cause toxic effects in the kidney.

### SUMMARY OF THE DISCLOSURE

The present disclosure relates to a conjugate of formula (I):

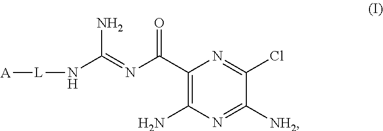

or a pharmaceutically acceptable salt thereof, wherein:

L is a bond,

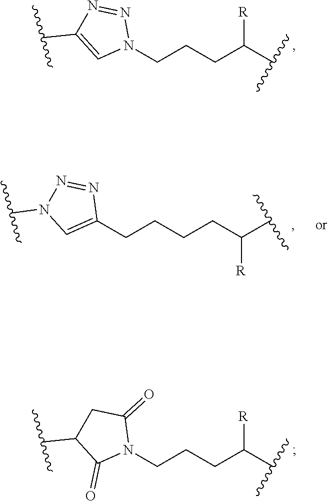

R is H or phenyl; and

A is a peptide designed to be retained in the lungs or a derivative thereof (each conjugate or a pharmaceutically acceptable salt being a “conjugate of the invention”).

In some embodiments, the peptide is ASHLRKLRKRL (SEQ ID NO: 1) or a derivative thereof. In some embodiments, the peptide comprises ASHLRKLRKRL (SEQ ID NO: 1) or a derivative thereof. In some embodiments, the peptide derivative is an acetylated, lipidated, amidated, derivatized with D-alanine, or derivatized with alpha-aminoisobutyric acid. In some embodiments, the peptide of SEQ ID NO: 1 comprises an additional leucine on the C-terminus (ASHLRKLRKRLL: SEQ ID NO: 59).

In some embodiments, the peptide is ASHLRKLRKRLL (SEQ ID NO: 59) or a derivative thereof. In some embodiments, the peptide comprises ASHLRKLRKRLL (SEQ ID NO: 59) or a derivative thereof.

In some embodiments, the peptide comprises SHLRKLRKRLL (SEQ ID NO: 58) or a derivative thereof.

In some embodiments, the peptide is any one of SEQ ID NOs: 2-13 and 56-57.

In some embodiments, the peptide is bound to L at the C-terminus. In some embodiments, the peptide is bound to L at the N-terminus. In some embodiments, the peptide is bound to L at an amino acid side chain residue of the peptide.

In some embodiments, the conjugate of the invention is selected from any one of SEQ ID NOs: 14-55.

The present disclosure also relates to a pharmaceutical composition comprising a conjugate of the invention and a pharmaceutically acceptable carrier or excipient (each composition being a “composition of the invention”).

The present disclosure also relates to a method for treating or preventing a disease or disorder in a subject in need thereof, comprising administering to the subject a therapeutically effective amount of the conjugate of the invention or a composition of the invention (each method being a “method of the invention”).

In some embodiments of the method of the invention, the disease or disorder is a pulmonary disease or disorder. In some embodiments, the pulmonary disease or disorder is cystic fibrosis, idiopathic pulmonary fibrosis, chronic obstructive pulmonary disease, asthma, emphysema, primary ciliary dyskinesia, pneumonia, or non-cystic fibrosis bronchiectasis.

In some embodiments of the method of the invention, the subject is a mammal. In some embodiments, the subject is a human.

In some embodiments of the method of the invention, the conjugate or the pharmaceutical composition is administered once a day. In some embodiments, the conjugate or the pharmaceutical composition is administered multiple times a day. In some embodiments, the conjugate is administered at a dose ranging from about 0.1 mg/kg to about 100 mg/kg.

In some embodiments of the method of the invention, the conjugate or the pharmaceutical composition is administered intranasally, intratracheally, intrapulmonary, intrabronchially, or by inhalation.

### BRIEF DESCRIPTION OF THE DRAWINGS

FIG. 1 shows the ENaC inhibition results of the illustrative conjugates of the invention in Ussing chamber.

FIG. 2 shows the stability assay results of the illustrative conjugates of the invention to cystic fibrosis lung protease.

FIG. 3 shows the stability assay results of the illustrative conjugates of the invention to cystic fibrosis lung protease.

FIG. 4A shows the pharmacokinetic profile of the illustrative conjugates of the invention. The results depict mean concentration by time on a semilog scale.

FIG. 4B shows the pharmacokinetic profile of the illustrative conjugates of the invention. The results depict mean concentration by time on a linear scale.

FIG. 5 shows efficacy of the ENaC inhibitor Conj. 13 in an in vivo model of cystic fibrosis. Control peptide (SPX-101) and 0.9% saline were used as a negative control. TMV, tracheal mucous velocity.

### DETAILED DESCRIPTION

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized enzyme-linked immunosorbent assay. As appropriate, procedures involving the use of commercially available kits and reagents are generally carried out in accordance with manufacturer defined protocols and/or parameters unless otherwise noted.

As used in the specification and the appended claims, the terms “a,” “an” and “the” include both singular and the plural referents unless the context clearly dictates otherwise.

The term “about” when immediately preceding a numerical value means±up to 20% of the numerical value. In some embodiments, “about” a numerical value means±up to 20%, ±up to 19%, ±up to 18%, ±up to 17%, ±up to 16%, ±up to 15%, ±up to 14%, ±up to 13%, ±up to 12%, ±up to 11%, ±up to 10%, ±up to 9%, ±up to 8%, ±up to 7%, ±up to 6%, ±up to 5%, ±up to 4%, ±up to 3%, ±up to 2%, ±up to 1%, ±up to less than 1%, or any other value or range of values therein, of the numerical value.

Throughout the present specification, numerical ranges are provided for certain quantities. These ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “polynucleotide” or “nucleic acid” refers to a polymer of nucleotide monomers covalently bonded in a chain. Exemplary nucleic acids include DNA and RNA.

The term “amino acid” refers to structural units (monomers) that make up a protein, polypeptide, or peptide. The amino acid can be a standard amino acid or a non-standard amino acid. Standard amino acids are alpha amino acids naturally used in the synthesis of polypeptides or proteins. Non-standard amino acids refer to amino acid derivatives or non-protein amino acids used in the synthesis of polypeptides or proteins. The term “polypeptide” or “protein” includes any polymer of amino acids or amino acid residues. A “peptide” is a small polypeptide of sizes less than about 15 to 20 amino acid residues. The term “amino acid sequence” refers to a series of amino acids or amino acid residues.

The term “derivative” as used herein refers to peptides that have been chemically modified, including, but not limited to, acetylation, ubiquitination, labeling, pegylation, lipidation, glycosylation, amidation, or addition of other molecules. These chemical modifications can be used, for example, to alter the pH or improve the molecule's solubility, absorption, or biological half-life, or decrease the toxicity of the molecule or eliminate or attenuate any undesirable side effects of the molecule. Chemical moieties capable of mediating such effects are disclosed in Remington's Pharmaceutical Sciences, 18th edition, A. R. Gennaro, Ed., Mack Publ., Easton, PA (1990), incorporated herein, by reference, in its entirety.

Methods for determining sequence similarity or identity between two or more nucleic acid sequences or two or more amino acid sequences are known in the art. Sequence similarity or identity may be determined using standard techniques, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection. Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). An exemplary BLAST program is the WU-BLAST-2 program which was obtained from Altschul et al., Methods in Enzymology, 266, 460-480 (1996); blast.wust1/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched: however, the values may be adjusted to increase sensitivity. Further, an additional algorithm is gapped BLAST as reported by Altschul et al. (1997) Nucleic Acids Res. 25, 3389-3402. Unless indicated otherwise, calculation of percent identity is performed in the instant disclosure using the BLAST algorithm available at the world wide web address: blast.ncbi.nlm.nih.gov/Blast.cgi.

As used herein, the terms “treat.” “treating,” or “treatment”, and grammatical variants thereof, have the same meaning as commonly understood by those of ordinary skill in the art. In some embodiments, these terms may refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (e.g. not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject in need of treatment may thus be a subject already afflicted with the disease or disorder in question. The terms “treat,” “treating.” or “treatment” includes inhibition or reduction of an increase in severity of a pathological state or symptoms relative to the absence of treatment, and is not necessarily meant to imply complete cessation of the relevant disease or condition. The terms “treat,” “treating,” or “treatment” can also refer to providing a therapeutically active agent, e.g., a caveolin-1 peptide or derivative thereof, to a biological sample obtained from a subject with a disease or disorder.

The term “pharmaceutically acceptable salt” includes both an acid and a base addition salt. Pharmaceutically acceptable salts can be obtained by reacting the compound of the invention functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Pharmaceutically acceptable salts can also be obtained by reacting a compound of the invention functioning as an acid, with an inorganic or organic base to form a salt, for example, salts of sodium, potassium, lithium, ammonium, calcium, magnesium, iron, zinc, copper, manganese, aluminum, ammonia, isopropylamine, trimethylamine, etc. In some embodiments, the pharmaceutically acceptable salt is a zinc salt. Those skilled in the art will further recognize that pharmaceutically acceptable salts can be prepared by reaction of the compounds of the invention with an appropriate inorganic or organic acid or base via any of a number of known methods.

As used herein, the term “pharmaceutically acceptable carrier or excipient” includes without limitation any adjuvant, carrier, glidant, sweetening agent, diluent, preservative, dye/colorant, flavor enhancer, surfactant, wetting agent, dispersing agent, suspending agent, stabilizer, isotonic agent, solvent, surfactant, and/or emulsifier. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

The term “interstitial lung disease” or “ILD” refers to a group of lung diseases affecting the interstitium (the tissue and space around the air sacs of the lungs). ILD can be classified according to a suspected or known cause, or can be idiopathic. For example, ILD can be classified as caused by inhaled substances (inorganic or organic), drug-induced (e.g., antibiotics, chemotherapeutic drugs, antiarrhythmic agents, statins), associated with connective tissue disease (e.g., systemic sclerosis, polymyositis, dermatomyositis, systemic lupus erythematous, rheumatoid arthritis), associated with pulmonary infection (e.g., atypical pneumonia, pneumocystis pneumonia, tuberculosis, Chlamydia trachomatis, Respiratory Syncytial Virus, COVID-19), associated with a malignancy (e.g., lymphangitic carcinomatosis), or can be idiopathic (e.g., sarcoidosis, idiopathic pulmonary fibrosis, Hamman-Rich syndrome, or antisynthetase syndrome).

The term “idiopathic pulmonary fibrosis” or “IPF” refers to a chronic, progressive form of lung disease characterized by fibrosis of the supporting framework (interstitium) of the lungs. Microscopically, lung tissue from patients having IPF shows a characteristic set of histologic/pathologic features known as usual interstitial pneumonia, characterized by a heterogeneous, variegated appearance with alternating areas of healthy lung, interstitial inflammation, fibrosis, and honeycomb change. By definition, the term IPF is used when the cause of the pulmonary fibrosis is unknown (“idiopathic”). Symptoms typically include gradual onset of shortness of breath and a dry cough. Other changes may include feeling tired, and abnormally large and dome shaped finger and toenails (nail clubbing). Complications may include pulmonary hypertension, heart failure, pneumonia, or pulmonary embolism.

The term “optimal dose” refers to an amount of therapeutically active agent effective to “alleviate” or “treat” a disease or disorder in a subject. An optimal dose of a therapeutically active agent may vary according to factors such as the disease state, age, sex, and weight of the individual. An optimal dose is also one in which any toxic or detrimental effects of the therapeutically active agent are outweighed by the therapeutically beneficial effects.

The compounds of the invention can have one or more asymmetric centers and can thus be enantiomers, racemates, diastereomers, other stereoisomers and mixtures thereof. The compounds of the invention include all such possible isomers (including geometric isomers), as well as their racemic and optically pure forms whether or not they are specifically depicted herein. Optically active (+) and (−), (R)- and(S)-, or (D)- and (L)-isomers can be prepared using chiral synthons or chiral reagents, or resolved using conventional techniques, for example, chromatography and fractional crystallization. Conventional techniques for the preparation or isolation of individual enantiomers include chiral synthesis from a suitable optically pure precursor or resolution of the racemate using, for example, chiral high pressure liquid chromatography (HPLC). When the compounds of the invention comprise an olefinic double bond or another center of geometric asymmetry, and unless specified otherwise, the compounds of the invention include both E and Z geometric isomers. Likewise, the compounds of the invention include all tautomeric forms.

An “effective amount” when used in connection with a conjugate of the invention means an amount of the compound of the invention that, when administered to a subject is effective in a method of the invention, alone or with another pharmaceutically active agent.

A “subject” is a human or non-human mammal, e.g., a bovine, horse, feline, canine, rodent, or non-human primate. The human can be a male or female, child, adolescent or adult. The female can be premenarcheal or postmenarcheal.

“Mammal” includes a human, domestic animal such as a laboratory animal (e.g., mouse, rat, rabbit, monkey, dog, etc.) and household pet (e.g., cat, dog, swine, cattle, sheep, goat, horse, rabbit), and a non-domestic, wild animal.

As used herein, the symbol “

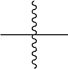

” (a “point of attachment bond”) denotes a bond that is a point of attachment between two chemical entities, one of which is depicted as being attached to the point of attachment bond and the other of which is not depicted as being attached to the point of attachment bond. For example, “

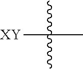

” indicates that the chemical entity “XY” is bonded to another chemical entity via the point of attachment bond.

### The Conjugates of the Invention

The present disclosure relates to a conjugate of formula (I):

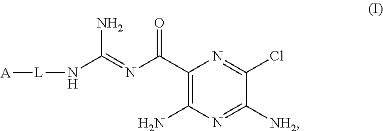

or a pharmaceutically acceptable salt thereof, wherein:

L is a bond,

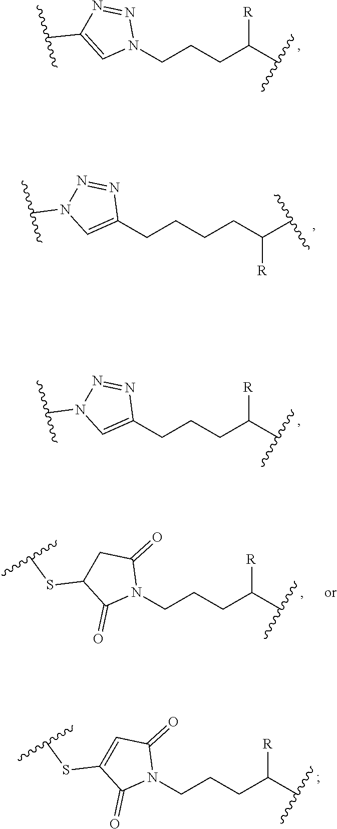

R is H or phenyl; and

A is a peptide designed to be retained in the lungs or a derivative thereof (e.g., peptides of Table 1).

The present disclosure relates to a conjugate of formula (I):

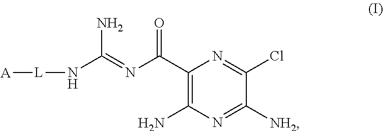

or a pharmaceutically acceptable salt thereof, wherein:

L is a bond,

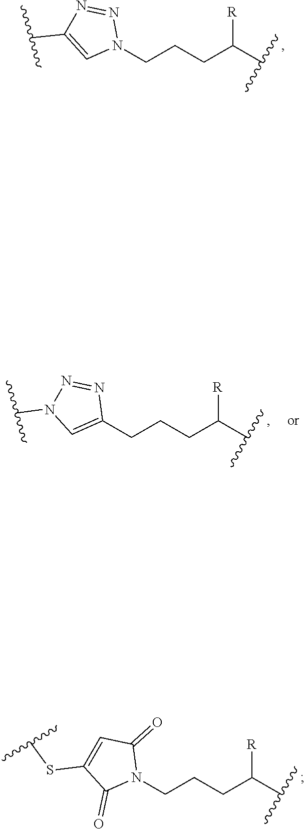

R is H or phenyl; and

A is a peptide designed to be retained in the lungs or a derivative thereof (e.g., peptides of Table 1).

In some embodiments of the conjugate of formula (I), L is a bond,

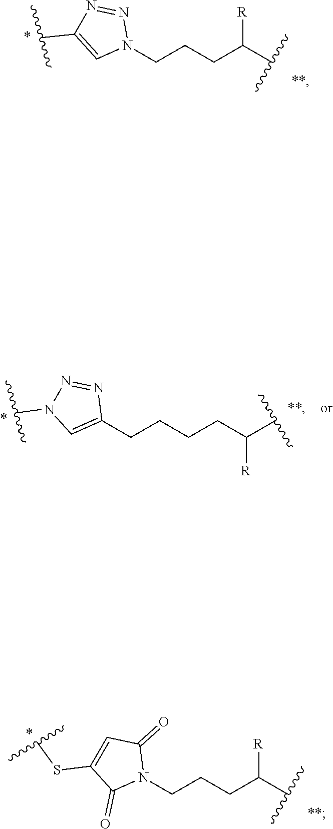

wherein * indicates the attachment point to A and ** indicates the attachment point to NH. In some embodiments of the conjugate of formula (I), L is a bond,

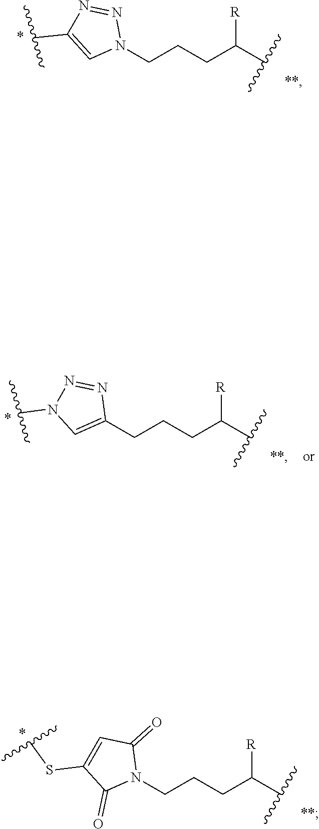

wherein * indicates the attachment point to A and ** indicates the attachment point to NH. In some embodiments, L is a bond. In some embodiments, L is

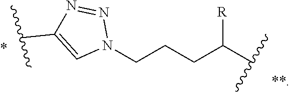

In some embodiments, L is

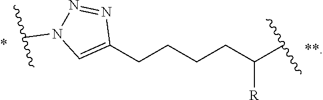

In some embodiments, L is

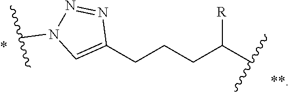

In some embodiments, L is

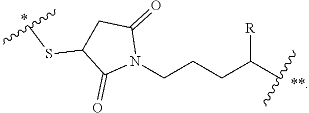

In some embodiments, L is

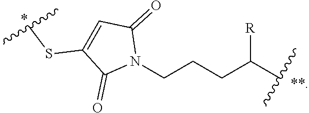

In some embodiments of the conjugate of formula (I), when L is

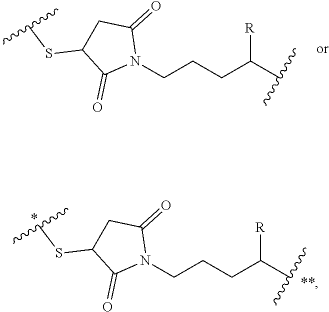

the sulfur atom is from a thiol residue of peptide A.

The present disclosure relates to a conjugate of formula (I):

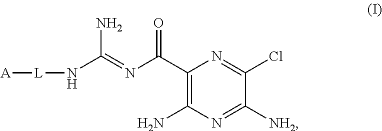

or a pharmaceutically acceptable salt thereof, wherein:

L is a bond,

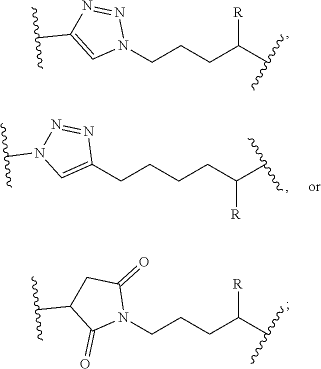

R is H or phenyl; and

A is a peptide designed to be retained in the lungs or a derivative thereof (e.g., peptides of Table 1).

In some embodiments of the conjugate of formula (I), L is a bond,

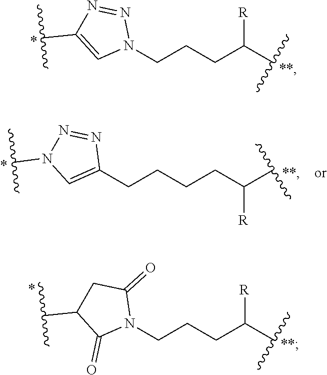

wherein * indicates the attachment point to A and ** indicates the attachment point to NH. In some embodiments, L is a bond. In some embodiments, L is

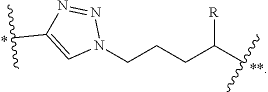

In some embodiments, L is

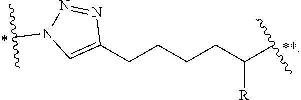

In some embodiments, L is

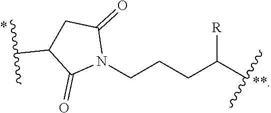

Each conjugate of formula (I) or a pharmaceutically acceptable salt being a “conjugate of the invention”

In some embodiments of the conjugate of formula (I), R is H. In some embodiments, R is phenyl. In some embodiments, R is unsubstituted phenyl.

In some embodiments of the conjugate of formula (I), the bond between A (the peptide designed to be retained in the lungs or a derivative thereof) and L is made through a side chain of an amino acid of A. In some embodiments, the bond between A and L is made through the N-terminus of A. In some embodiments, the bond between A and L is made through the C-terminus of A. In some embodiments, in forming the bond between A and L, one atom (such as H) or a chemical group of A is replaced with a covalent bond to L.

The peptides of the present disclosure are designed to be retained in the lungs or derivatives thereof. The peptides can be synthetic, recombinant, or chemically modified peptides isolated or generated using methods well known in the art. In some embodiments, the peptide or derivative thereof comprises one or more hydrophobic amino acids (e.g., valine, leucine, or isoleucine). In some embodiments, the peptide or derivative thereof comprises one or more non-standard amino acids (e.g., an amino acid with a chemically modified side chain or a D-amino acid). In some embodiments, the non-standard amino acid is D-alanine, alpha-aminoisobutyric acid, hydroxyproline, epsilon-azido-lysine, 6-aminohexanoic acid, and/or propargylglycine. In some embodiments, the non-standard amino acid is a beta amino acid. In some embodiments, the non-standard amino acid is an analog of alanine, glycine, valine, or leucine, including, but not limited to, tert-butyloxycarbonyl (Boc)-6-aminohexanoic acid, Boc-L-alpha,beta-diaminopropionic acid, Boc-L-propargylglycine, Boc-beta-cyclohexyl-L-alanine, Di-Fmoc-L-alpha,beta-diaminopropionic acid, 9-fluorenylmethoxycarbonyl (Fmoc)-(N-beta-(2,4-dinitrophenyl))-L-alpha,beta-diaminopropionic acid, Fmoc-(N-beta-Boc)-D-alpha,beta-diaminopropionic acid, Fmoc-(N-beta-Boc)-L-alpha,beta-diaminopropionic acid, Fmoc-(N-beta-allyloxycarbonyl)-L-alpha,beta-diaminopropionic acid, Fmoc-(N-gamma-4-methyltrityl)-L-alpha.gamma-diaminobutyric acid, Fmoc-(N-gamma-Boc)-L-alpha.gamma-diaminobutyric acid, Fmoc-4-fluoro-L-phenylglycine, Fmoc-5,5,5-trifluoro-DL-leucine, Fmoc-Dab (Dde)-OH, Fmoc-L-2-amino-3-(dimethylamino)-propionic acid, Fmoc-L-2-aminocaproic acid, Fmoc-L-allylglycine, Fmoc-L-alpha-t-butylglycine, Fmoc-alpha-aminoisobutyric acid, Fmoc-beta-(2-pyridyl)-L-alanine, Fmoc-beta-(3-pyridyl)-L-alanine, Fmoc-beta-cyclopropyl-L-alanine, and Fmoc-beta-t-butyl-L-alanine. In some embodiments, the peptide or derivative thereof comprises a modification on the N-terminus (e.g., acetylation or acylation), C-terminus (e.g., amidation), or internally.

In some embodiments, the peptides of the present disclosure are designed to be resistant to enzymatic cleavage. In some embodiments, the peptides of the present disclosure are designed to be resistant to lung proteases (e.g., neutrophil elastase). In some embodiments, the peptides are synthetic, recombinant, or chemically modified peptides isolated or generated to be resistant to enzymatic cleavage. In some embodiments, the peptide or derivative thereof comprises one or more non-standard amino acids (e.g., an amino acid with a chemically modified side chain, a D-amino acid, or a beta-amino acid) to prevent enzymatic cleavage and/or increase stability. In some embodiments, the peptide or derivative thereof comprises D-alanine to prevent enzymatic cleavage and/or increase stability. In some embodiments, the peptide or derivative thereof comprises alpha-aminoisobutyric acid to prevent enzymatic cleavage and/or increase stability.

In some embodiments, the peptide or derivative thereof comprises one or more mutations, e.g., an insertion, deletion, or substitution of amino acids. In some embodiments, the peptide or derivative thereof comprises one or more amino acid substitutions to a non-standard amino acid, e.g., an amino acid with a chemically modified side chain or a D-amino acid.

In some embodiments of the conjugate of formula (I), the peptide is ASHLRKLRKRL (SEQ ID NO: 1) or a derivative thereof. In some embodiments of the conjugate of formula (I), the peptide comprises ASHLRKLRKRL (SEQ ID NO: 1) or a derivative thereof. In some embodiments of the conjugate of formula (I), the peptide comprises ASHLRKLRKRL (SEQ ID NO: 1) with an additional leucine on the C-terminus (i.e., ASHLRKLRKRLL: SEQ ID NO: 59). In some embodiments, peptide derivative is an acetylated, lipidated, and/or amidated SEQ ID NO: 1. In some embodiments, the peptide derivative is SEQ ID NO: 1 which is acetylated, lipidated, amidated, derivatized with hydroxyl proline, derivatized with 6-aminohexanoic acid, derivatized with epsilon-azido-lysine, derivatized with D-alanine, derivatized with alpha-aminoisobutyric acid, and/or derivatized with propargylglycine. In some embodiments, the peptide derivative includes one or two amino acid substitutions. In some embodiments, the peptide derivative of SEQ ID NO: 1 comprises a peptide where L-Ala (A) of SEQ ID NO: 1 is substituted with another amino acid. In some embodiments, the substitution is with a hydrophobic amino acid, beta amino acid, natural amino acid, or unnatural amino acid. In some embodiments, the peptide derivative of SEQ ID NO: 1 comprises a peptide where L-Ala (A) of SEQ ID NO: 1 is substituted with D-alanine or alpha-aminoisobutyric acid.

In some embodiments of the conjugate of formula (I), the peptide is SHLRKLRKRLL (SEQ ID NO: 58) or a derivative thereof. In some embodiments of the conjugate of formula (I), the peptide comprises SHLRKLRKRLL (SEQ ID NO: 58) or a derivative thereof. In some embodiments, peptide derivative is an acetylated, lipidated, and/or amidated SEQ ID NO: 58. In some embodiments, the peptide derivative includes one or two amino acid substitutions. In some embodiments, the substitution is with a hydrophobic amino acid, beta amino acid, natural amino acid, or unnatural amino acid.

In some embodiments of the conjugate of formula (I), the peptide is ASHLRKLRKRLL (SEQ ID NO: 59) or a derivative thereof. In some embodiments of the conjugate of formula (I), the peptide comprises ASHLRKLRKRLL (SEQ ID NO: 59) or a derivative thereof. In some embodiments, peptide derivative is an acetylated, lipidated, and/or amidated SEQ ID NO: 59. In some embodiments, the peptide derivative is SEQ ID NO: 59 which is acetylated, lipidated, amidated, derivatized with hydroxyl proline, derivatized with 6-aminohexanoic acid, derivatized with epsilon-azido-lysine, derivatized with D-alanine, derivatized with alpha-aminoisobutyric acid, and/or derivatized with propargylglycine. In some embodiments, the peptide derivative includes one or two amino acid substitutions. In some embodiments, the peptide derivative of SEQ ID NO: 59 comprises a peptide where L-Ala (A) of SEQ ID NO: 59 is substituted with another amino acid. In some embodiments, the substitution is with a hydrophobic amino acid, beta amino acid, natural amino acid, or unnatural amino acid. In some embodiments, the peptide derivative of SEQ ID NO: 59 comprises a peptide where L-Ala (A) of SEQ ID NO: 59 is substituted with D-alanine or alpha-aminoisobutyric acid.

In some embodiments of the conjugate of formula (I), the peptide or derivative thereof is any one of SEQ ID NOs: 2-13 and 56-57. In some embodiments, the peptide derivative is any one of SEQ ID NOs: 2-13 and 56-57 which is acetylated, lipidated, amidated, derivatized with hydroxyl proline, derivatized with 6-aminohexanoic acid, derivatized with epsilon-azido-lysine, derivatized with D-alanine, derivatized with alpha-aminoisobutyric acid, and/or derivatized with propargylglycine.

Exemplary amino acid sequences of the peptide or derivatives thereof are shown below in Table 1. The term “Ac” refers to an acetyl group; the term “NH2” refers to an amino group; the term “Hyp” refers to a hydroxy proline; the term “Ahx” refers to a 6-aminohexanoic acid; the term “AzK” refers to an epsilon-azido-lysine; the term “Aib” refers to alpha-aminoisobutyric acid, the term “D-Ala” refers to D-alanine; and the term “Pgy” refers to a propargylglycine.

TABLE 1
Peptides designed to be retained in
the lungs and derivatives thereof

| SEQ ID NO: | Sequence |
|---|---|
| 1 | ASHLRKLRKRL |
| 2 | Ac-K-(Ahx)-ALA(Hyp)YI-NH2 |
| 3 | Ac-(AzK)-Ahx-ALA(Hyp)YI-NH2 |
| 4 | Ac-(Pgy)-Ahx-ALA(Hyp)YI-NH2 |
| 5 | Ac-C-Ahx-ALA(Hyp)YI-NH2 |
| 6 | Ac-K-(Ahx)-ASHLRKLRKRLL-NH2 |
| 7 | Ac-(AzK)-(Ahx)-ASHLRKLRKRLL-NH2 |
| 8 | Ac-(Pgy)-(Ahx)-ASHLRKLRKRLL-NH2 |
| 9 | Ac-C-(Ahx)-ASHLRKLRKRLL-NH2 |
| 10 | Ac-K-(Ahx)-VSKRR-NH2 |
| 11 | Ac-(AzK)-(Ahx)-VSKRR-NH2 |
| 12 | Ac-(Pgy)-(Ahx)-VSKRR-NH2 |
| 13 | Ac-C-(Ahx)-VSKRR-NH2 |
| 56 | Ac-K-(Aib)-SHLRKLRKRLL-NH2 |
| 57 | Ac-K-(D-Ala)-SHLRKLRKRLL-NH2 |
| 58 | SHLRKLRKRLL |
| 59 | ASHLRKLRKRLL |

In some embodiments, the peptide or derivative thereof comprises or consists of the amino acid sequence of any one of SEQ ID NOs: 1-13 and 56-59. In some embodiments, the peptide or derivative thereof comprises the amino acid sequence of any one of SEQ ID NOs: 1-13 and 56-59 with one or more mutations relative thereto. For example, in some embodiments, the peptide or derivative thereof comprises 1, 2, 3, 4, 5, 6, or more mutations relative to any one of SEQ ID NOs: 1-13 and 56-59.

In some embodiments, the peptide comprises or consists of the amino acid sequence of SEQ ID NO: 1. In some embodiments, the peptide comprises the amino acid sequence of SEQ ID NO: 1 with one or more mutations relative thereto. For example, in some embodiments, the peptide comprises 1, 2, 3, 4, 5, 6, or more mutations relative to SEQ ID NO: 1.

In some embodiments, the peptide comprises or consists of the amino acid sequence of SEQ ID NO: 58. In some embodiments, the peptide comprises the amino acid sequence of SEQ ID NO: 58 with one or more mutations relative thereto. For example, in some embodiments, the peptide comprises 1, 2, 3, 4, 5, 6, or more mutations relative to SEQ ID NO: 58.

In some embodiments, the peptide comprises or consists of the amino acid sequence of SEQ ID NO: 59. In some embodiments, the peptide comprises the amino acid sequence of SEQ ID NO: 59 with one or more mutations relative thereto. For example, in some embodiments, the peptide comprises 1, 2, 3, 4, 5, 6, or more mutations relative to SEQ ID NO: 59.

In some embodiments of the conjugate of formula (I), the peptide or a derivative thereof is bound to L at the N-terminus. In some embodiments, the peptide or a derivative thereof is bound to L at the C-terminus. In some embodiments, the peptide or a derivative thereof is bound to L at an amino acid side chain residue of the peptide.

In some embodiments of the conjugate of formula (I), the conjugate is selected from any one of SEQ ID NOs: 14-55. In Table 2, the term “Ac” refers to an acetyl group: the term “NH2” refers to an amino group: the term “Hyp” refers to a hydroxy proline: the term “Ahx” refers to a 6-aminohexanoic acid: the term “AzK” refers to an epsilon-azido-lysine: the term “Pgy” refers to a propargylglycine: the term “Aib” refers to alpha-aminoisobutyric acid, the term “D-Ala” refers to D-alanine, and amiloride and benzamil refer to ENaC inhibitors having the structures depicted below with or without L in formula (I).

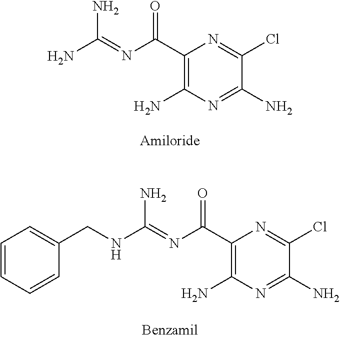 [SMILES: NC(N)=NC(=O)c1nc(Cl)c(N)nc1N ; N/C(=N\C(=O)c1nc(Cl)c(N)nc1N)NCc1ccccc1]

“Amililoride” or “Benzamil” in Table 2 can have the following specific structures denoted as Amil_{AC}, Amil_{CC1}, Amil_{CC2}, Amil_{TC}, Benz_{AC}, Benz_{CC1}, Benz_{CC2}, or Benz_{TC} as shown below:

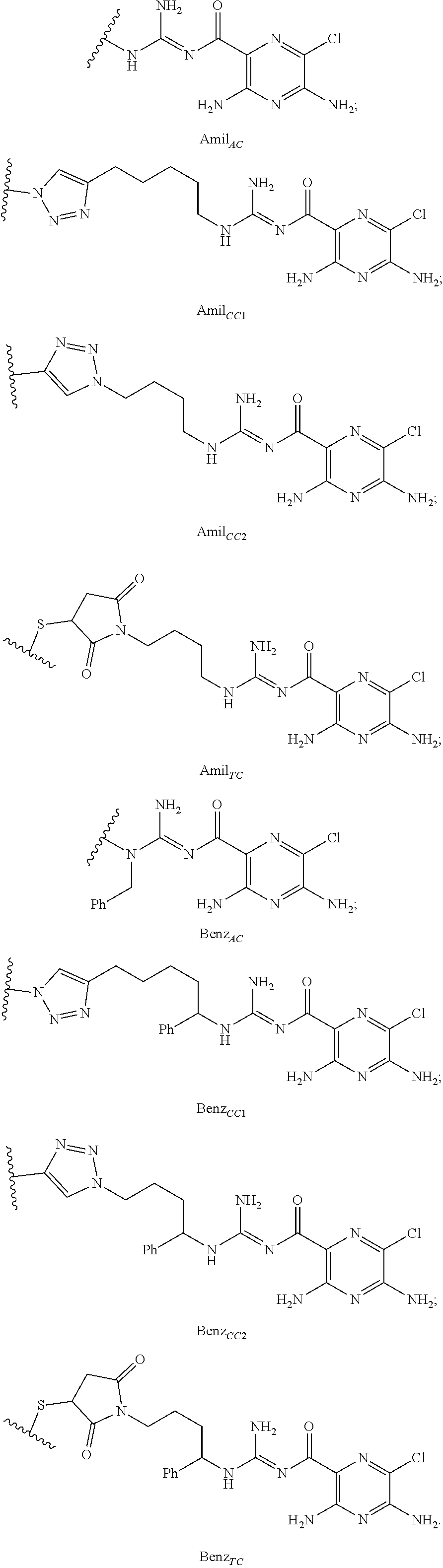

In above structures of Amil_{TC} and Benz_{TC}, the sulfur atom is from a side chain residue of the peptide, such as Cys residue.

TABLE 2

| SEQ | |
|---|---|
| ID NO: | Sequence |
| 14 | Amiloride-(Ahx)-ALA(Hyp)YI-NH2 |
| 15 | Ac-(amiloride)K-(Ahx)-ALA(Hyp)YI-NH2 |
| 16 | Ac-(amiloride-AzK)-Ahx-ALA(Hyp)YI-NH2 |
| 17 | Ac-(amiloride-Pgy)-Ahx-ALA(Hyp)YI-NH2 |
| 18 | Ac-(amiloride-C)-Ahx-ALA(Hyp)YI-NH2 |
| 19 | Ac-(benzamil-AzK)-Ahx-ALA(Hyp)YI-NH2 |
| 20 | Ac-(benzamil)-Pgy)-Ahx-ALA(Hyp)YI-NH2 |
| 21 | Ac-(benzamil-C)-Ahx-ALA(Hyp)YI-NH2 |
| 22 | Amiloride-(Ahx)-ASHLRKLRKRLL-NH2 |
| 23 | Ac-(amiloride)K-(Ahx)-ASHLRKLRKRLL-NH2 |
| 24 | Ac-(amiloride-AzK)-Ahx-ASHLRKLRKRLL-NH2 |
| 25 | Ac-(amiloride-Pgy)-Ahx-ASHLRKLRKRLL-NH2 |
| 26 | Ac-(amiloride-C)-Ahx-ASHLRKLRKRLL-NH2 |
| 27 | Ac-(amiloride-C)-Ahx-VSKRR-NH2 |
| 28 | Ac-(benzamil-Pgy)-Ahx-ASHLRKLRKRLL-NH2 |
| 29 | Ac-(benzamil-C)-Ahx-ASHLRKLRKRLL-NH2 |
| 30 | Amiloride-(Ahx)-VSKRR-NH2 |
| 31 | Ac-(amiloride)K-(Ahx)-VSKRR-NH2 |
| 32 | Ac-(amiloride-AzK)-Ahx-VSKRR-NH2 |
| 33 | Ac-(amiloride-Pgy)-Ahx-VSKRR-NH2 |
| 34 | Ac-(amiloride-C)-Ahx-VSKRR-NH2 |
| 35 | Ac-(benzamil-AzK)-Ahx-VSKRR-NH2 |
| 36 | Ac-(benzamil-Pgy)-Ahx-VSKRR-NH2 |
| 37 | Ac-(benzamil-C)-Ahx-VSKRR-NH2 |
| 53 | Ac-(amiloride)-K-(Aib)-SHLRKLRKRLL-NH2 |
| 54 | Ac-(benzamil)-K-(Aib)-SHLRKLRKRLL-NH2 |
| 55 | Ac-(amiloride)-K-(D-Ala)-SHLRKLRKRLL-NH2 |
| 38 | Ac-(Amil_{AC})K-(Ahx)-ALA(Hyp)YI-NH2 |
| 39 | Ac-(Amil_{AC})K-(Ahx)-ASHLRKLRKRLL-NH2 |
| 40 | Ac-(Amil_{AC})K-(Ahx)-VSKRR-NH2 |
| 41 | Ac-(Amil_{CC1}-AzK)-Ahx-ALA(Hyp)YI-NH2 |
| 42 | Ac-(Amil_{CC1}-AzK)-Ahx-ASHLRKLRKRLL-NH2 |
| 43 | Ac-(Amil_{CC1}-AzK)-Ahx-VSKRR-NH2 |
| 44 | Ac-(Amil_{CC2}-Pgy)-Ahx-ALA(Hyp)YI-NH2 |
| 45 | Ac-(Amil_{CC2}-Pgy)-Ahx-ASHLRKLRKRLL-NH2 |
| 46 | Ac-(Amil_{CC2}-Pgy)-Ahx-VSKRR-NH2 |
| 47 | Ac-(Amil_{TC}-C)-Ahx-ALA(Hyp)YI-NH2 |
| 48 | Ac-(Amil_{TC}-C)-Ahx-ASHLRKLRKRLL-NH2 |
| 49 | Ac-(Amil_{TC}-C)-Ahx-VSKRR-NH2 |
| 50 | Ac-(Amil_{CC1})-K-(Aib)-SHLRKLRKRLL-NH2 |
| 51 | Ac-(Benz_{CC1})-K-(Aib)-SHLRKLRKRLL-NH2 |
| 52 | Ac-(Amil_{CC1})-K-(D-Ala)-SHLRKLRKRLL-NH2 |

In some embodiments, the conjugates of the invention comprise N- and/or C-terminal modifications. In some embodiments, the conjugates of the invention comprise an N-terminal modification, e.g., acylation or acetylation. In some embodiments, the conjugates of the invention comprise a C-terminal modification, e.g., amidation.

In some embodiments, the conjugate of the invention comprises a peptide comprising ASHLRKLRKRL (SEQ ID NO: 1). In some embodiments, the conjugate of the invention is selected from any one of SEQ ID NOs: 22-26 and 28-29.

In some embodiments, the conjugate of the invention comprises a peptide comprising SHLRKLRKRLL (SEQ ID NO: 58). In some embodiments, the conjugate of the invention is selected from any one of SEQ ID NOs: 50-55.

In some embodiments, the conjugate of the invention comprises a peptide comprising ASHLRKLRKRLL (SEQ ID NO: 59). In some embodiments, the conjugate of the invention is selected from any one of SEQ ID NOs: 22-26 and 28-29.

In some embodiments, the conjugate of SEQ ID NO: 15 is the conjugate of SEQ ID NO: 38. In some embodiments, the conjugate of SEQ ID NO: 16 is the conjugate of SEQ ID NO: 41. In some embodiments, the conjugate of SEQ ID NO: 17 is the conjugate of SEQ ID NO: 44. In some embodiments, the conjugate of SEQ ID NO: 18 is the conjugate of SEQ ID NO: 47. In some embodiments, the conjugate of SEQ ID NO: 23 is the conjugate of SEQ ID NO: 39. In some embodiments, the conjugate of SEQ ID NO: 24 is the conjugate of SEQ ID NO: 42. In some embodiments, the conjugate of SEQ ID NO: 25 is the conjugate of SEQ ID NO: 45. In some embodiments, the conjugate of SEQ ID NO: 26 is the conjugate of SEQ ID NO: 48. In some embodiments, the conjugate of SEQ ID NO: 27 is the conjugate of SEQ ID NO: 49. In some embodiments, the conjugate of SEQ ID NO: 31 is the conjugate of SEQ ID NO: 40. In some embodiments, the conjugate of SEQ ID NO: 32 is the conjugate of SEQ ID NO: 43. In some embodiments, the conjugate of SEQ ID NO: 33 is the conjugate of SEQ ID NO: 46. In some embodiments, the conjugate of SEQ ID NO: 50 is the conjugate of SEQ ID NO: 53. In some embodiments, the conjugate of SEQ ID NO: 51 is the conjugate of SEQ ID NO: 54. In some embodiments, the conjugate of SEQ ID NO: 52 is the conjugate of SEQ ID NO: 55.

In some embodiments, the conjugates of the invention have improved absorption across epithelial barriers like the gut and airways, compared to the peptide designed to be retained in the lungs or a derivative thereof by itself. In some embodiments, the conjugates of the invention have longer half-lives, compared to the peptide designed to be retained in the lungs or a derivative thereof by itself. In some embodiments, the conjugates of the invention are retained in the lung for a longer period of time, compared to the peptide designed to be retained in the lungs or a derivative thereof by itself. In some embodiments, the conjugates of the invention have higher lung retention, compared to amiloride or benzamil. In some embodiments, the conjugates of the invention are resistant to enzymatic cleavage by lung proteases (e.g., neutrophil elastase).

In some embodiments, greater than about 50% of the conjugates of the invention remains in tact (uncleaved) after being incubated PBS and neutrophil elastase for 60 minutes at 37° C. In some embodiments, greater than about 60% of the conjugates of the invention remains in tact after being incubated PBS and neutrophil elastase for 60 minutes at 37° C. In some embodiments, greater than about 70% of the conjugates of the invention remains in tact after being incubated PBS and neutrophil elastase for 60 minutes at 37° C. In some embodiments, greater than about 75% of the conjugates of the invention remains in tact after being incubated PBS and neutrophil elastase for 60 minutes at 37° C. In some embodiments, greater than about 80% of the conjugates of the invention remains in tact after being incubated PBS and neutrophil elastase for 60 minutes at 37° C. In some embodiments, greater than about 85% of the conjugates of the invention remains in tact after being incubated PBS and neutrophil elastase for 60 minutes at 37° C. In some embodiments, greater than about 90% of the conjugates of the invention remains in tact after being incubated PBS and neutrophil elastase for 60 minutes at 37° C. In some embodiments, the presence of in-tact conjugate is measured by HPLC.

In some embodiments, the conjugates of the invention inhibit ENaC. In some embodiments, the conjugates of the invention is a more potent ENaC inhibitor than amiloride. In some embodiments, the conjugates of the invention provides IC₅₀ for the ENaC inhibition of less than about 1 μM, less than about 0.9 μM, less than about 0.8 μM, less than about 0.7 μM, less than about 0.6 μM, less than about 0.5 μM, less than about 0.4 μM, less than about 0.3 μM, less than about 0.2 μM, or less than about 0.1 μM. In some embodiments, the conjugates of the invention provides IC₅₀ for the ENaC inhibition that is at least 2-fold more potent than amiloride. In some embodiments, the conjugates of the invention provides IC₅₀ for the ENaC inhibition that is at least 5-fold more potent than amiloride. In some embodiments, the conjugates of the invention provides IC₅₀ for the ENaC inhibition that is at least 7-fold more potent than amiloride. In some embodiments, the conjugates of the invention provides IC₅₀ for the ENaC inhibition that is at least 8-fold more potent than amiloride. In some embodiments, the conjugates of the invention provides IC₅₀ for the ENaC inhibition that is at least 9-fold more potent than amiloride. In some embodiments, the conjugates of the invention provides IC₅₀ for the ENaC inhibition that is at least 10-fold more potent than amiloride.

Without bound to any theory, the improved potency of the conjugates of the invention compared to amiloride may be attributed, in part, to conformation change of the conjugate in association with ENaC after the conjugate binds to the active site. The additional conformation change step can impact the K_{I} (inhibition constant) which is similar to K_{D} (equilibrium dissociation constant), a calculated ratio of kₒₙ (association constant) and k_{off} (dissociation constant) between ENaC target and the ENac inhibitor.

$${K}_{D} ≅ {K}_{I}$$

$${K}_{D} = {k}_{off} / {k}_{on}$$

For amiloride, it is believed that dissociation and association to ENaC is a one-step process.

$$[ E N a C ] + [ Amil ] {{{k}_{on}}_{⇌}}_{{k}_{off}} [ ENaC : A m i l ]$$

Whereas for the conjugates of the invention, it is hypothesized that the conformation change step is added to the equilibrium.

$$[ E N a C ] + [ Conj . ] {{{k}_{1}}_{⇌}}_{{k}_{2}} [ ENaC ] - [ Conj . ] {{{k}_{3}}_{⇌}}_{{k}_{4}} [ ENaC : Conj . ]$$

The dissociation constant (k_{off}) for this two-step process is:

K_{off}=k₂k₄/(k₂+k₃+k₄)

Due to the confirmation change, the dissociation constant (k_{off}) of this two-step equilibrium can be greater, leading to a higher K_{D} (i.e., higher K₁).

In some embodiments, the conjugates of the invention comprises a peptide having at least 10 amino acids. In some embodiments, the conjugates of the invention comprises a peptide having at least 11 amino acids. In some embodiments, the conjugates of the invention comprises a peptide having at least 12 amino acids. In some embodiments, the peptide is “A” in formula (I) (e.g., a peptide designed to be retained in the lungs or a derivative thereof).

In some embodiments, the conjugates of the invention comprises a peptide which forms a helical structure having a hydrophobic side. In some embodiments, the conjugates of the invention comprises a peptide which forms a helical structure having a positively charged side. In some embodiments, the conjugates of the invention comprises a peptide which forms a helical structure having a hydrophobic side and a positively charged side.

### The Composition of the Invention

The present disclosure also relates a pharmaceutical composition comprising a conjugate of the invention and a pharmaceutically acceptable carrier or excipient.

In some embodiments, the pharmaceutically acceptable carrier or excipient is an adjuvant, carrier, glidant, sweetening agent, diluent, preservative, dye/colorant, flavor enhancer, surfactant, wetting agent, dispersing agent, suspending agent, stabilizer, isotonic agent, solvent, surfactant, and/or emulsifier. In some embodiments, the pharmaceutically acceptable carrier or excipient is a sugar (such as lactose, glucose, sucrose, and trehalose), starch (such as corn starch and potato starch), cellulose and its derivatives (such as sodium carboxymethyl cellulose, ethyl cellulose and cellulose acetate), tragacanth, malt, gelatin, talc, cocoa butter, wax, animal and vegetable fat, paraffin, silicon, bentonite, silicic acid, zinc oxide, oil (such as peanut oil, cottonseed oil, safflower oil, sesame oil, olive oil, corn oil and soybean oil), glycols (such as propylene glycol), polyol (such as glycerin, sorbitol, mannitol and polyethylene glycol), ester (such as ethyl oleate and ethyl laurate), agar, buffering agent (such as magnesium hydroxide and aluminum hydroxide), alginic acid, pyrogen-free water, saline solution, Ringer's solution, ethyl alcohol, phosphate buffer solution, and the like, or combinations thereof, and any other compatible substances employed in pharmaceutical formulations.

In some embodiments, solid pharmaceutical carrier is starch, lactose, trehalose, calcium sulfate dihydrate, terra alba, sucrose, talc, gelatin, agar, pectin, acacia, magnesium stearate or stearic acid, and the like, or combinations thereof. In some embodiments, liquid pharmaceutical carrier is syrup, peanut oil, olive oil, saline, phosphate buffer solution, water, dextrose, glycerol, and the like, or combinations thereof. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Gennaro, A R, Remington: The Science and Practice of Pharmacy, Lippincott Williams & Wilkins Publishers: 21st Ed, 2005 (or latest edition).

In some embodiments, the composition of the invention is prepared for administration orally, parenterally, sublingually, transdermally, intravitreally, rectally, transmucosally, topically, via inhalation, via buccal administration, intrapleurally, intravenously, intraarterially, intragastrically, intraperitoneally, subcutaneously, intramuscularly, intranasally, intratracheally, intrapulmonary, intrabronchially, intrathecally, or intraarticularly. In some embodiments, the composition of the invention is prepared for administration by lung instillation. In some embodiments, the composition of the invention is prepared for administration by a nebulizer, dry powder inhaler, or metered dose inhaler.

In some embodiments, the composition of the invention is prepared for topical ocular administration. In some embodiments, the composition for topical ocular administration is an aqueous solution. In some embodiments, the composition for topical ocular administration is a semi-solid composition, e.g., a viscous or semi-viscous gel.

In some embodiments, the composition of the invention can deliver the conjugate of the invention in an amount ranging from about 0.01 mg/kg to about 250 mg/kg, from about 0.01 mg/kg to about 100 mg/kg, from 0.01 mg/kg to about 50 mg/kg, or from 0.05 mg/kg to about 50 mg/kg, or any subranges therebetween.

### The Methods of the Invention

The present disclosure also relates to a method for treating or preventing a disease or disorder in a subject in need thereof, comprising administering to the subject a therapeutically effective amount of the conjugate of the invention or a composition of the invention.

In some embodiments, the disease or disorder is a pulmonary disease or disorder. In some embodiments, the pulmonary disease or disorder is cystic fibrosis, idiopathic pulmonary fibrosis, chronic obstructive pulmonary disease, asthma, emphysema, primary ciliary dyskinesia, pneumonia, or non-cystic fibrosis bronchiectasis. In some embodiments, chronic obstructive pulmonary disease is chronic bronchitis, asthma and bronchiectasis. In some embodiments, the pulmonary disease or disorder is cystic fibrosis.

In some embodiments, the disease or disorder is a cardiovascular disease or disorder. In some embodiments, the cardiovascular disease or disorder is hypertension or congestive heart failure.

In some embodiments, the disease or disorder is hyperaldosteronism.

In some embodiments, the disease or disorder is a skin condition. In some embodiments, the skin condition is psoriasis, eczema, atopic dermatitis, or ichthyosis.

In some embodiments, the disease or disorder is an ocular disease or disorder. In some embodiments, the ocular disease or disorder is dry eye syndrome.

In some embodiments, the disease or disorder is cirrhosis, nephrotic syndrome, or hypokalemia.

In some embodiments of the methods of the invention, the subject is mammal. In some embodiments, the subject is a human.

In some embodiments, the conjugate of the invention or the composition of the invention increases mucociliary clearance in a subject in need thereof. In some embodiments, the conjugate of the invention or the composition of the invention reduces or decreases thick mucus, phlegm, or sputum in a subject in need thereof. In some embodiments, the conjugate of the invention or the composition of the invention reduces lung infection in a subject in need thereof. In some embodiments, the conjugates of the invention or the composition of the invention improves lung function in a subject in need thereof. In some embodiments, the subject in need thereof has cystic fibrosis.

In some embodiments of the methods of the invention, the conjugate of the invention or the composition of the invention is administered once a day, twice a day, three times a day, or more. In some embodiments, the conjugate of the invention or the composition of the invention is administered once a day. In some embodiments, the conjugate of the invention or the composition of the invention is administered multiple times a day. In some embodiments, the conjugate of the invention or the composition of the invention is administered once per week, twice per week, three times per week, four times per week, five times per week, once per month, twice per month, three times per month, once every two months, once every three months, once every six months, or once per year.

In some embodiments of the methods of the invention, the conjugate of the invention or the composition of the invention is administered at a dose ranging from about 0.01 mg/kg to about 250 mg/kg, from about 0.01 mg/kg to about 100 mg/kg, from about 0.01 mg/kg to about 50 mg/kg, or from about 0.05 mg/kg to about 50 mg/kg, or any subranges therebetween. In some embodiments, the conjugate of the invention or the composition of the invention is administered at a dose of about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg.

In some embodiments of the methods of the invention, the conjugate of the invention or the composition of the invention is administered orally, parenterally, sublingually, transdermally, rectally, transmucosally, intravitreally, topically, via inhalation, via buccal administration, intrapleurally, intravenously, intraarterially, intragastrically, intraperitoneally, subcutaneously, intramuscularly, intranasally, intratracheally, intrapulmonary, intrabronchially, intrathecally, or intraarticularly. In some embodiments, the conjugate of the invention or the composition of the invention is administered intranasally, intratracheally, intrapulmonary, intrabronchially, or by inhalation. In some embodiments, the conjugate of the invention or the composition of the invention is administered by a nebulizer, dry powder inhaler, or metered dose inhaler.

### Examples

### General Synthesis

The conjugates of the invention can be prepared using known coupling and conjugation methods, including but not limited to the following methods.

Click Chemistry: A copper catalyzed reaction of substituted azido compounds are coupled with alkynes to produce 1,4-disubstituted 1,2,3-triazoles with high efficiency as shown in Scheme 1. In Scheme 1, R₁ can either be the peptide designed to be retained in the lungs or a derivative, or the amiloride analog and R₂ can be the appropriate pairing molecule for conjugation.

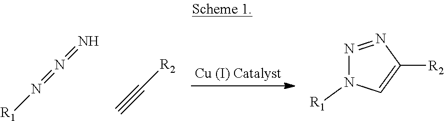

Preparation of Allyl-amiloride (R═H) or benzamil (R═Ph) derivatives lead to peptide conjugates as shown below, from coupling with azido-peptides.

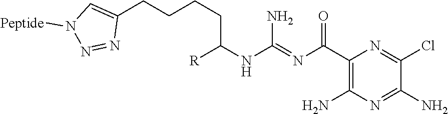

Conversely, allyl-peptides can be coupled with azido-amiloride (R═H) or azido-benzamil (R-Ph) using Click Chemistry to produce conjugates as shown below.

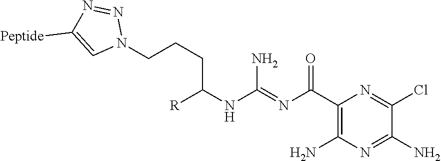

Maleimide Chemistry: Another approach to preparation of amiloride or benzamil conjugates can be achieved using maleimide chemistry. In this case, a thiol is included in the peptide as cysteine and the peptide is coupled to maleimido-amiloride (R═H) or benzamil (R═Ph) as shown in Scheme 2.

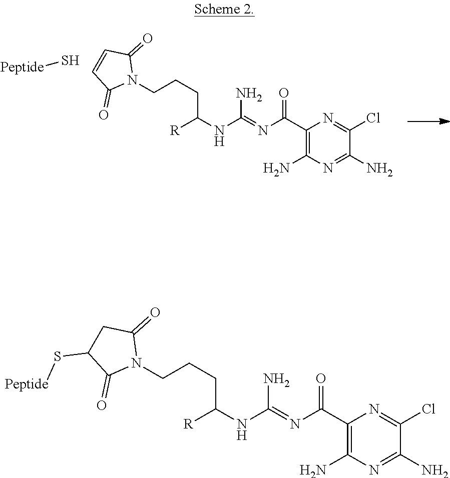

Amine Coupling: Another method to complete the preparation of amiloride containing peptides is through direct coupling of amiloride to free amines in the peptide as illustrated in Scheme 3.

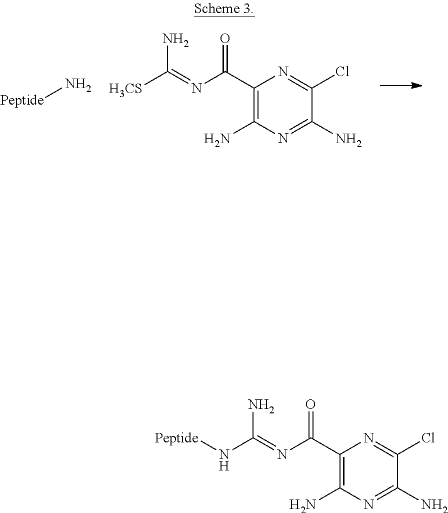

### Synthesis of Intermediates

Intermediates useful in the preparation of the conjugates of the invention are listed in Table 3, which can be prepared by methods disclosed in Schemes 4-14.

TABLE 3
Structures of Intermediates

| Intermediate ID | Structure |
|---|---|
| TM1 | 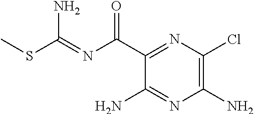 [SMILES: CS/C(N)=N/C(=O)c1nc(Cl)c(N)nc1N] |
| TM2 | 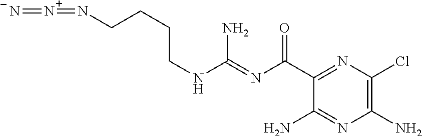 [SMILES: [N-]=[N+]=NCCCCN/C(N)=N/C(=O)c1nc(Cl)c(N)nc1N] |
| TM3 | 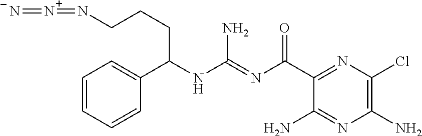 [SMILES: [N-]=[N+]=NCCCC(N/C(N)=N/C(=O)c1nc(Cl)c(N)nc1N)c1ccccc1] |
| TM4 | 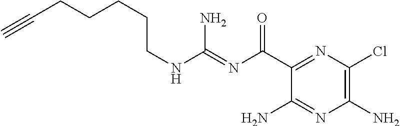 [SMILES: C#CCCCCCN/C(N)=N/C(=O)c1nc(Cl)c(N)nc1N] |
| TM5 | 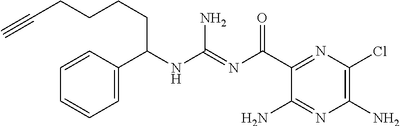 [SMILES: C#CCCCCC(N/C(N)=N/C(=O)c1nc(Cl)c(N)nc1N)c1ccccc1] |
| TM6 | 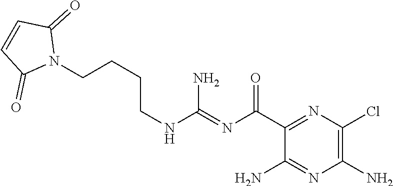 [SMILES: N/C(=N\C(=O)c1nc(Cl)c(N)nc1N)NCCCCN1C(=O)C=CC1=O] |
| TM7 | 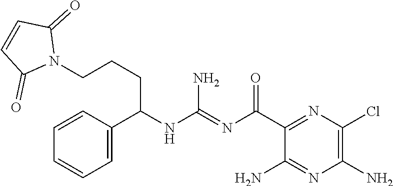 [SMILES: N/C(=N\C(=O)c1nc(Cl)c(N)nc1N)NC(CCCN1C(=O)C=CC1=O)c1ccccc1] |
| I1 | 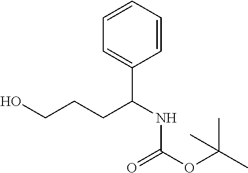 [SMILES: CC(C)(C)OC(=O)NC(CCCO)c1ccccc1] |
| ITM1 | 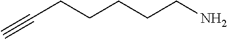 |
| ITM3 | 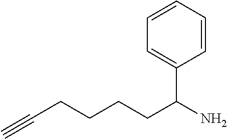 |
| ITM5 | 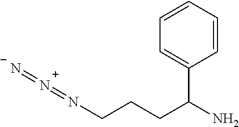 [SMILES: [N-]=[N+]=NCCCC(N)c1ccccc1] |
| ITM7 | 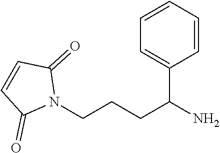 [SMILES: NC(CCCN1C(=O)C=CC1=O)c1ccccc1] |

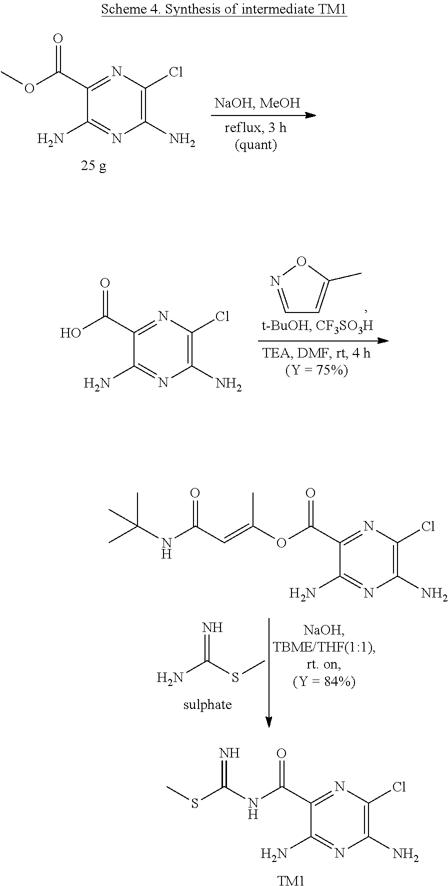

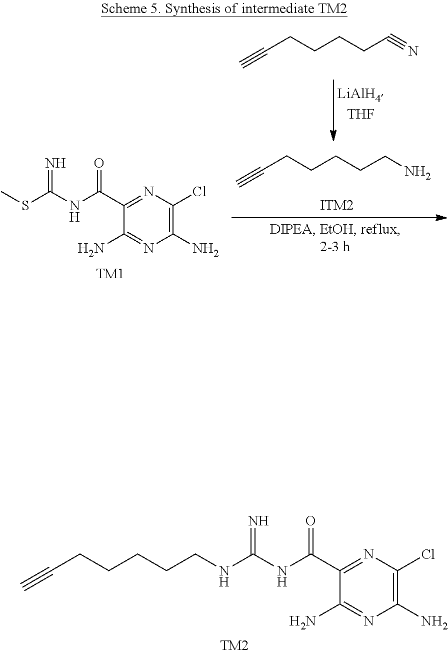

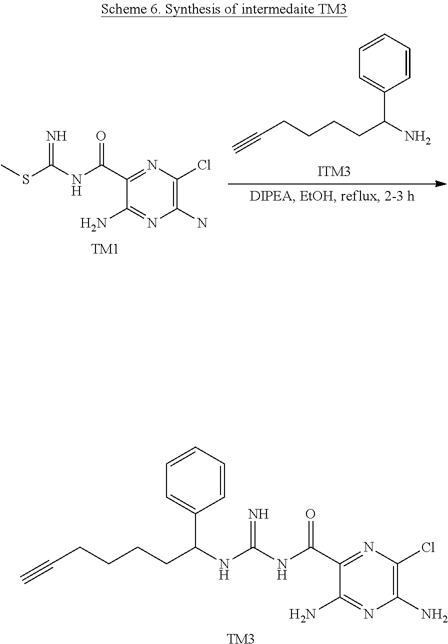

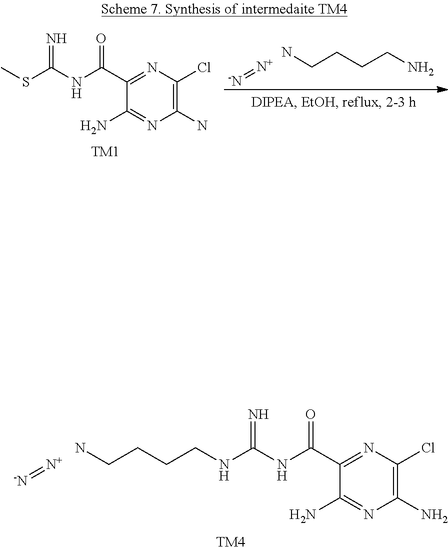

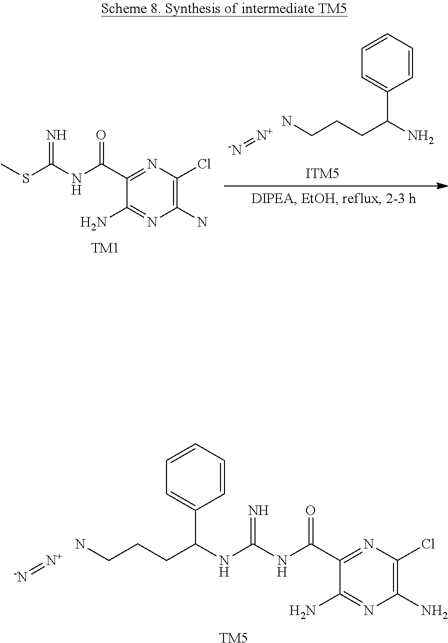

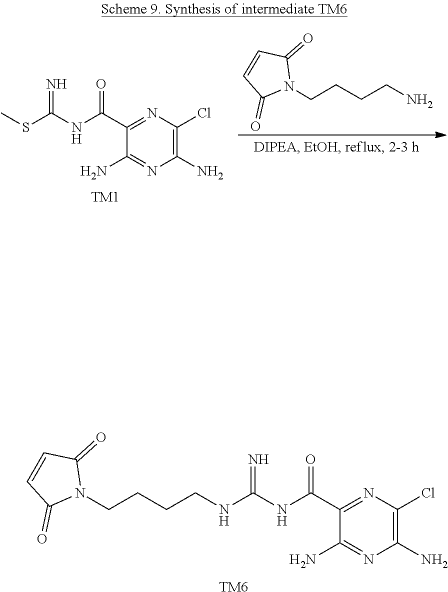

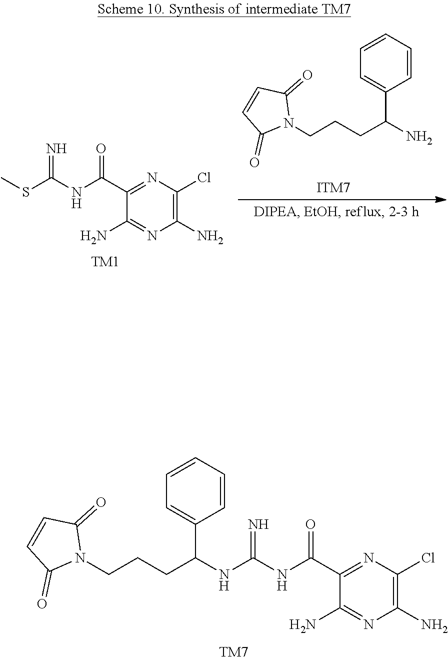

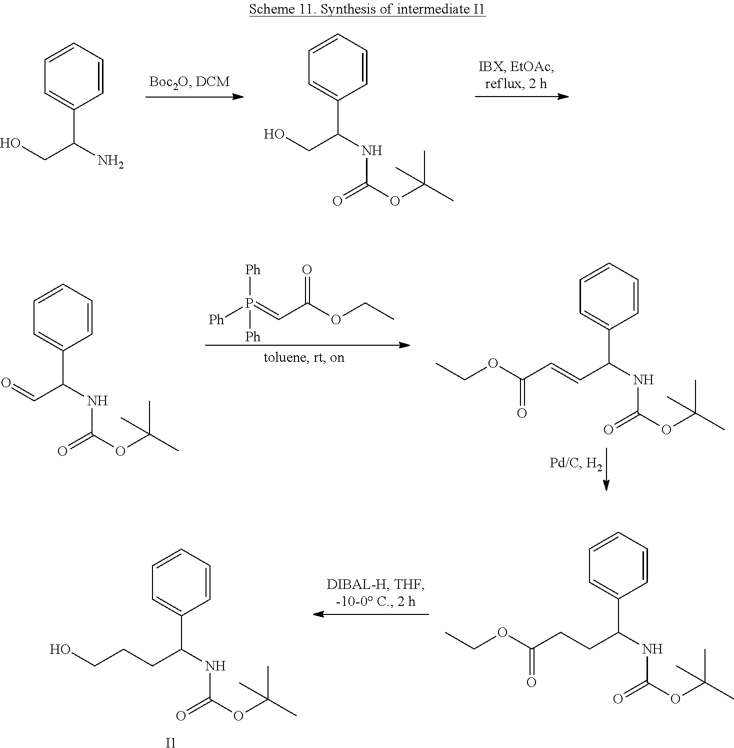

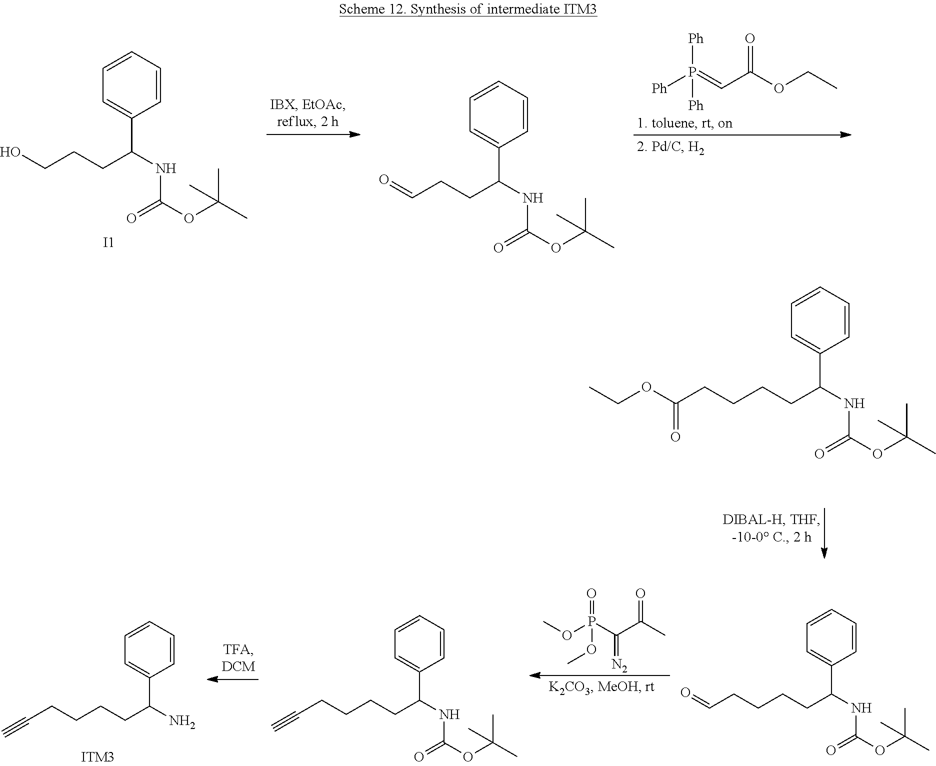

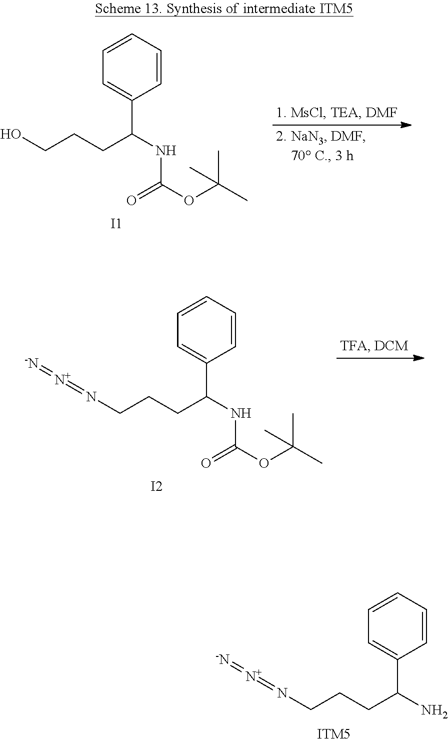

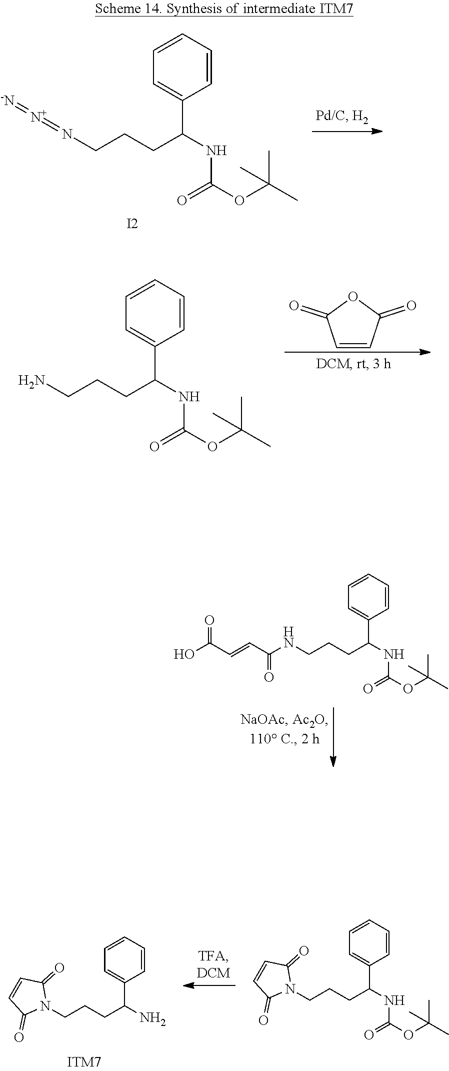

### Example 1. Synthesis of Conjugates of the Invention

Conjugate Nos. 1-12 were prepared according to the above described chemical synthesis methods, including amine coupling, thiol-maleimide conjugation, and click chemistry using the peptides described herein (e.g., peptides of Table 1). The conjugate number with the associated chemistry method and sequence identification number are shown in Table 4.

TABLE 4

| Conjugate Identity | | |
|---|---|---|
| or Control | Chemistry Method | SEQ ID NO: |
| 1 | Amine-coupling | 38 |
| 2 | Amine-coupling | 39 |
| 3 | Amine-coupling | 40 |
| 4 | Click chemistry | 41 |
| 5 | Click chemistry | 42 |
| 6 | Click chemistry | 43 |
| 7 | Click chemistry | 44 |
| 8 | Click chemistry | 45 |
| 9 | Click chemistry | 46 |
| 10 | Thiol-maleimide conjugation | 47 |
| 11 | Thiol-maleimide conjugation | 48 |
| 12 | Thiol-maleimide conjugation | 49 |
| 13 | Click chemistry | 50 |
| 14 | Click chemistry | 51 |
| 15 | Click chemistry | 52 |
| Control 1 | Peptide only control | 2 |
| Control 2 | Peptide only control | 6 |
| Control 3 | Peptide only control | 10 |

### Example 2: ENaC Inhibition by the Illustrative Conjugates of the Invention

The objective of this study was to measure the ability of the illustrative conjugates to modulate ion transport function of ENAC in normal human bronchial epithelial (NHBE) cell monolayers. Sodium transport function of ENAC expressed in NHBE monolayers was evaluated using an Ussing epithelial voltage clamp apparatus. See, Hirsh et al., J. Pharmacol. Exp. Ther. 2008; 325:77-88, hereby incorporated by reference in its entirety.

### Cell Culture Procedures

Primary NHBE cells were isolated from bronchi of normal healthy patients, expanded in Lonza media (BEGM™ BulletKit™), and plated on Snapwell™ filters to form an epithelium. Cells on Snapwell™ inserts were grown for at least 21 days in Vertex Differentiation media and exposed to an air-liquid interface to promote differentiation. At least one day before the assay, mucous overlaying the apical surface was removed from the inserts by incubating the apical surface for about 30 minutes with ≥200 μL of differentiation media followed by aspiration of the mucous film and media.

### Electrophysiological Methods

The NHBE cell monolayers grown on Snapwell™ filter inserts were transferred to Physiologic Instruments Ussing recording chambers (Physiologic Instruments, Inc., San Diego, CA) and superfused with HB-PS. One or more 6-channel or 8-channel Physiologic Instruments VCC MC6 or VCC MC8 epithelial voltage clamps were used in combination to record short circuit current (I_{SC}) from up to 24 inserts simultaneously. Air was used to operate the bubble lifts to rapidly circulate solutions in each Ussing chamber half and to aerate the monolayers.

Inserts were voltage clamped at 0 m V to record the I_{SC}. Bumetanide (20 μM) was added to the basolateral side of all inserts. Test conjugates prepared as DMSO (Sigma-Aldrich) stocks were added cumulatively and sequentially to the apical Ussing chamber halves with at least 3 minutes between each addition. Negative and positive control groups were treated with vehicle (HB-PS+≤1% DMSO, Charles River Laboratories Cleveland, Inc.) and amiloride (Sigma-Aldrich), respectively.

Amiloride (30 μM) or benzamil (10 μM, Sigma-Aldrich) was added to the apical side of all Snapwell™ filter inserts (test conjugate and control treated) after the highest test conjugate concentration in order to inhibit all ENaC current and establish the maximum ENaC inhibition response. Time and volume matched additions of DMSO to the basolateral side of the test conjugate treated inserts and both sides of the vehicle treated inserts was made to maintain osmotic balance across the epithelia. Transepithelial resistance was monitored with small voltage steps. The assay was performed at 35±2° C.

### Data Analysis

ENaC current at each test conjugate concentration was calculated as the peak current change produced by each test conjugate treated insert minus the mean current change produced by the corresponding DMSO additions to time-matched vehicle controls inserts. Cumulative difference data was fit to a Hill equation of the form:

$${I}_{EnaC} = \{ [ {I}_{EnaC ( Max )} - {{I}_{EnaC}}_{( Min )} ] - \{ [ {I}_{EnaC ( Max )} - {{I}_{EnaC}}_{( Min )} ] / [ 1 + {( [ T e s t ] / I C 50 )}^{N} ] \} \} + {I}_{EnaC ( Min )}$$

Where I_{EnaC} is the I_{SC} peak difference current measured at each test conjugate concentration [Test]. IC₅₀ is the peptide conjugate concentration at half maximal inhibition, N is the Hill coefficient, I_{EnaC}(Max) is the maximum amiloride or benzamil inhabitable I_{SC} difference current, I_{EnaC} (Min) is the minimum I_{SC} difference current in the absence of EnaC modulators.

### Results

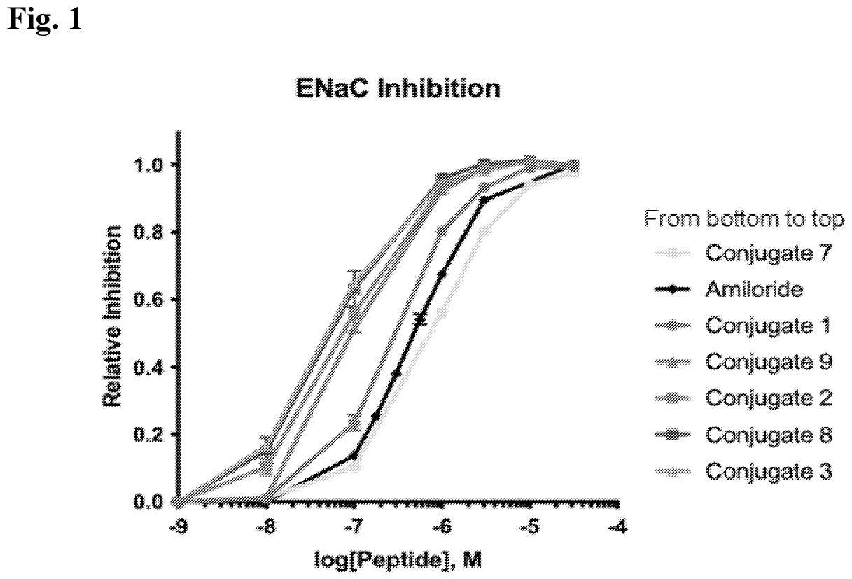

As shown in FIG. 1, the test conjugates demonstrated 100% inhibition of ENaC. The IC₅₀ for the ENaC inhibition is shown in Table 5. Conjugate nos. 3, 5, and 8 were about 10-fold more potent than amiloride.

TABLE 5

| Conjugate Identity | | |
|---|---|---|
| or Control | SEQ ID NO. | IC₅₀ (μM) |
| Amiloride (control) | N/A | 0.36-0.67 |
| 1 | 38 | 0.29 |
| 2 | 39 | 0.08 |
| 3 | 40 | 0.05 |
| 4 | 41 | 0.24 |
| 5 | 42 | 0.03 |
| 6 | 43 | 0.09 |
| 7 | 44 | 0.79 |
| 8 | 45 | 0.06 |
| 9 | 46 | 0.10 |
| 10 | 47 | 0.49 |
| 11 | 48 | 0.07 |
| 12 | 49 | 0.08 |
| Peptide only Control 1 | 2 | Did not inhibit |
| Peptide only Control 2 | 6 | Did not inhibit |
| Peptide only Control 3 | 10 | Did not inhibit |

### Example 3. Stability of the Illustrative Conjugates of the Invention to Neutrophil Elastase

The peptide backbone of a majority of the conjugates comprises a neutrophil elastase cleavage site. Experiments were therefore performed to determine the stability of the conjugates to protease cleavage.

The conjugates of the invention were incubated in PBS or PBS and 0.1 mg neutrophil elastase for 60 minutes at 37° C. The conjugates were then run on HPLC to determine their stability against protease cleavage.

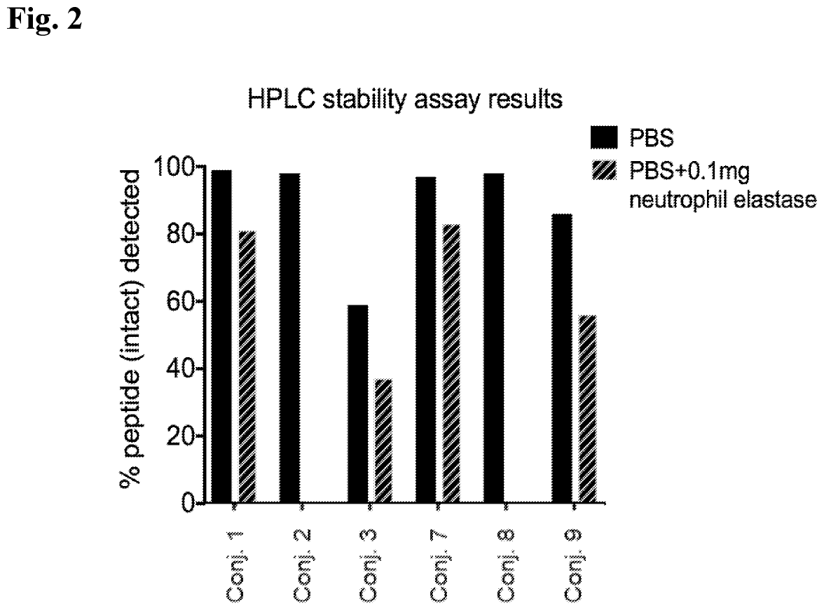

As shown in FIG. 2, all peptide conjugates tested were susceptible to cleavage by neutrophil elastase with Conj. 2 and Conj. 8 being the most susceptible.

Further experiments were performed to modify or remove the protease cleavage site from the peptide conjugates by replacing L-alanine with alpha-aminoisobutyric acid (Aib) or D-alanine (D-Ala). The newly synthesized peptide conjugates are shown in Table 6 below.

TABLE 6

| Conjugate | | SEQ |
|---|---|---|
| Identity | Peptide Conjugate Sequence | ID NO. |
| 13 | Ac-(Amil_{CC1})-K-(Aib)-SHLRKLRKRL | 50 |
| | L-NH2 | |
| 14 | Ac-(Benz_{CC1})-K-(Aib)-SHLRKLRKRL | 51 |
| | L-NH2 | |
| 15 | Ac-(Amil_{CC1})-K-(D-Ala)-SHLRKLRK | 52 |
| | RLL-NH2 | |

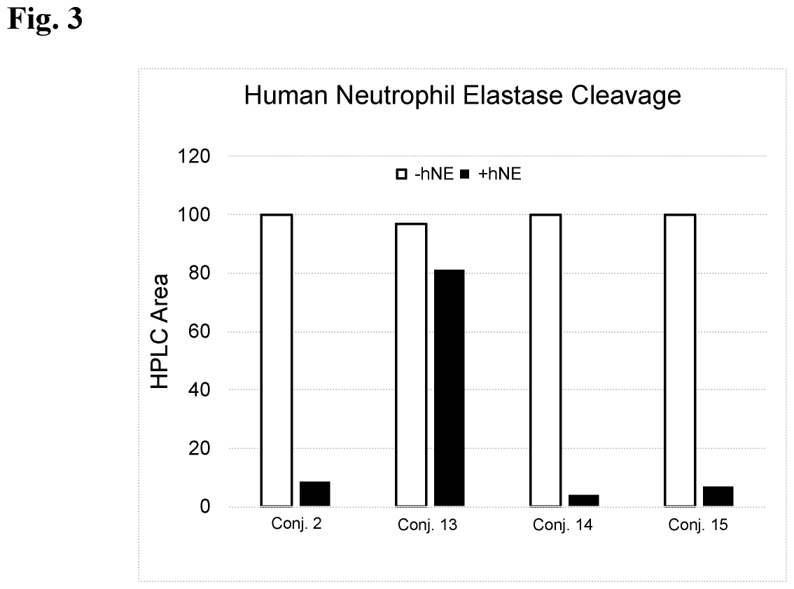

As shown in FIG. 3, Conj. 13 had drastically reduced susceptibility to cleavage by neutrophil elastase compared to Conj. 2, Conj. 14, and Conj. 15. These results demonstrate that amiloride peptide conjugates that substitute L-alanine with alpha-aminoisobutyric acid prevented cleavage by neutrophil elastase and increased stability of the peptide conjugates.

### Example 4. Pharmacokinetics of the Illustrative Conjugates of the Invention

The pharmacokinetic profile of the conjugates of the present invention was determined in blood and lung tissue of rats.

### Experimental Procedures

Peptide Conj. 8 (SEQ ID NO: 45) and Conj. 9 (SEQ ID NO: 46) were formulated in 0.9% saline and intratracheally administered to 5-7 week-old rats (Sprague Dawley; male; 224-250 g). The pharmacokinetic profile of the peptide conjugates was then determined in blood and lung tissue. The study design is shown in Table 7 below.

TABLE 7

| | | No. | | | Route | | Dose | |
|---|---|---|---|---|---|---|---|---|
| | No. of | animals/ | | | of | Dose | Vol. | Test |
| Group | animals | subgroup | Subgroup | Sex | Admin | (mg/kg) | (mL/kg) | Article |
| 1 | 6 | 3 | A | M | IT | 5 | 1.5 | Conj. 8 |
| | | 3 | B | | | | | |
| 2 | 6 | 3 | A | M | IT | 5 | 1.5 | Conj. 9 |
| | | 3 | B | | | | | |

For subgroup A, whole blood samples were collected from animals at 0.083, 0.25, and 0.5 hours following drug administration and for subgroup B, whole blood samples were collected at 0.75, 1, and 2 hours following drug administration for plasma analysis. Lung and bronchoalveolar lavage fluid (BALF) were collected at 0.5 and 2 hours following drug administration.

Whole blood was collected by tail nick at the predetermined time points and then maximal volume by cardiac stick for the final time point. The plasma was spun and collected within 30 minutes of collection. Upon euthanasia of the animal, a necropsy was performed and lungs harvested and weighed for analysis. After weighing the lungs, BALF was collected with 0.5 mL of 0.9% saline. The lungs were then reweighed after BALF collection. Lung, BALF, and plasma samples were frozen until analysis.

Frozen lung tissue was homogenized using a Precellys Evolution tabletop homogenizer (3×6500 rpm) with 4× dilution using normal rat plasma. 50 μL aliquots of homogenized lung tissue was then transferred into a 96 well plate for extraction with methanol. Harvested BALF was transferred in 25 μL aliquots into a 96 well plate and diluted with 25 μL of rat plasma before extraction with methanol. Plasma samples were transferred in 50 μL aliquots into a 96 well plate for extraction with methanol.

Standards were prepared by producing a working stock solution of Conj. 8 and Conj. 9 at 1 mg/mL. The Conj. 8 assay was performed using Conj. 9 as an internal standard at 1000 ng/mL. The Conj. 9 assay was performed using Conj. 8 as an internal standard at 1000 ng/mL.

Extraction was performed by direct precipitation with 3 volumes (150 μL) of methanol containing 1000 ng/ml of Conj. 8 and Conj. 9 as an internal standard. The plate was vortexed, followed by centrifugation (10 minutes at 4° C. and 2500 g) and dried down under heated nitrogen. Reconstitution was in 100 μL of initial mobile phase A (0.1% TFA: 0.1% FA in water) with 5% of mobile phase B (CAN, 0.1% TFA: 0.1% FA), before placing in an autosampler.

Plasma, lung, and BALF samples were analyzed for Conj. 8 and Conj. 9 by liquid chromatography with tandem mass spectrometry (LC/MS/MS) (API 4000; Kinetex® 2.6 μm C18 Column 50×2.1 mm; column temp.=50° C.; mobile phase A-0.1% TFA: 0.1% FA in water; mobile phase B-ACN, 0.1% TFA: 0.1% FA; flow rate-0.25 mL/min). Pharmacokinetic parameters were derived using sparse sampling noncompartmental analysis methods of Phoenix WinNonLin® version 8.0 (Certara USA, Inc.).

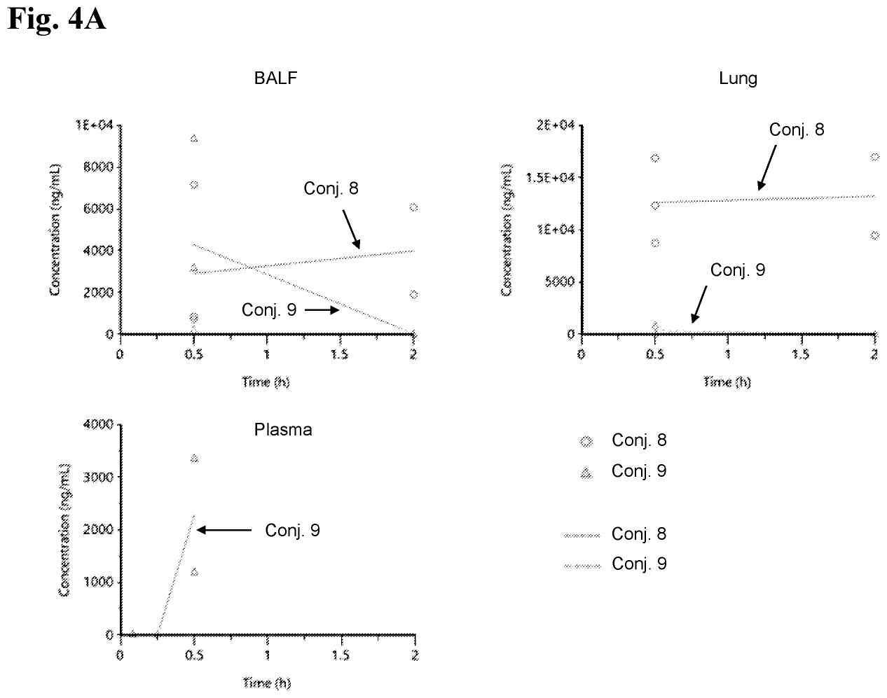

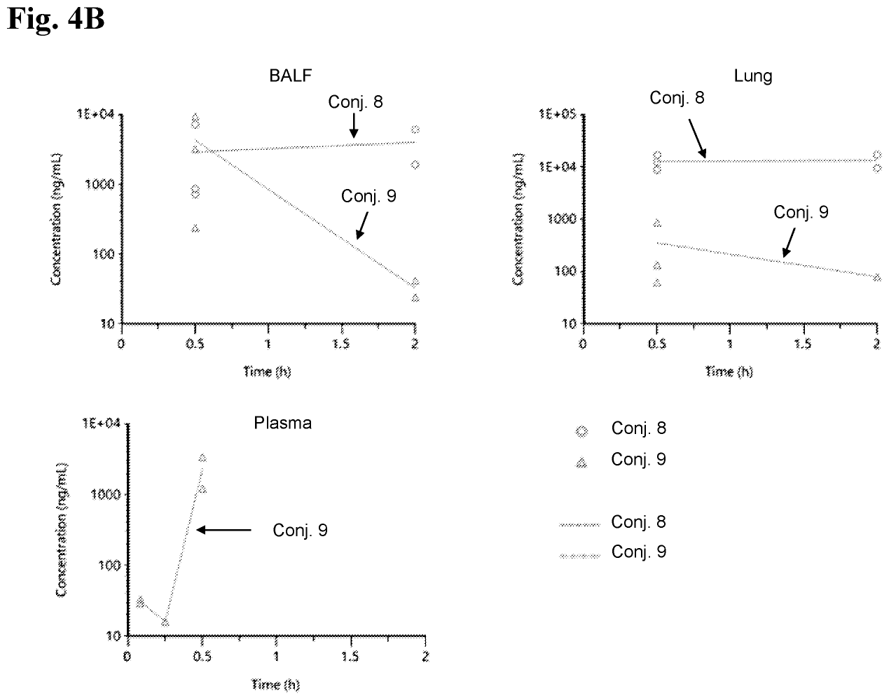

The pharmacokinetic profile of the test conjugates are shown in FIGS. 4A-4B and Table 8. Tₘₐₓ, the time after dosing at which the maximum concentration was observed; Cₘₐₓ, the maximum observed concentration measured after dosing; AUCₗₐₛₜ, the area under the concentration versus time curve from the start of dose administration to the last observed quantifiable concentration calculated using the log/linear trapezoidal method; and MRTₗₐₛₜ, mean residence time as defined by AUMCₗₐₛₜ/AUCₗₐₛₜ.

TABLE 8

| Conjugate | Dose Level | | Cₘₐₓ | Tₘₐₓ | AUCₗₐₛₜ | MRTₗₐₛₜ |
|---|---|---|---|---|---|---|
| Identity | (mg/kg) | Tissue | (ng/mL) | (h) | (h*ng/mL) | (h) |
| Conj. 8 | 5 | BALF | 3990 | 2 | 5910 | 1.26 |
| | | Lungs | 13200 | 2 | 22500 | 1.16 |
| | | Plasma | NC | NC | NC | NC |
| Conj. 9 | 5 | BALF | 4290 | 0.5 | 2380 | 0.663 |
| | | Lungs | 352 | 0.5 | 362 | 0.932 |
| | | Plasma | 2290 | 0.5 | 293 | 0.492 |

NC: Not calculated due to all values below the limit of quantitation

Conj. 8 concentrations were blank in all plasma samples, indicating that there was no absorption of Conj. 8 from the lungs into the blood above the limit of quantitation of 25 ng/mL. Lung tissues had an approximate mean Cₘₐₓ concentration of 13,000 ng/ml at both 0.5 h and 2 h, which was >3 to 4-fold higher than the BALF concentration, which was 2910 ng/ml and 3990 ng/mL at 0.5 h and 2 h, respectively (Table 8, FIGS. 4A-4B). These data indicate that the drug remains in the lung and is not detectable in systemic circulation.

Conj. 9 exhibited a Cₘₐₓ concentration that was relatively high in BALF and plasma and low in lung tissues at 4290 ng/mL, 2290 ng/ml, and 352 ng/ml, respectively (Table 8, FIGS. 4A-B). The low lung tissue concentrations and high BALF and plasma concentrations suggests that Conj. 9 is more likely to move from lung tissue into systemic circulation.

### Example 5. Efficacy of the Illustrative Conjugates of the Invention

The conjugates of the present invention were tested for their ability to increase lung mucociliary clearance in a sheep model of cystic fibrosis.

### Experimental Procedures

The peptide conjugates of the present invention were tested in vivo using a sheep model of cystic fibrosis as described in Scott et al., Am J. Resp. Crit. Care Med., 2017; 196 (6): 734-744; and Sabater et al., PLOS ONE, 2019; 14 (11): e0224764, hereby incorporated by reference in their entirety.

Briefly, tracheal mucous velocity (TMV) was determined in sheep by insufflation of radio-opaque Teflon disks into the trachea and measuring the movement of the disks over a 1 minute period by scintigraphy.

Intubated sheep were treated with the CFTR inhibitor INH-172 by nebulization, which caused a reduction in the mucus transport rate in the trachea. Once a baseline inhibited TMV was obtained at 4 hours following CFTR inhibition, then ENaC inhibitor conjugate (Conj. 13) or control was nebulized into the sheep lung and TMV measurements were repeated hourly.

### Results

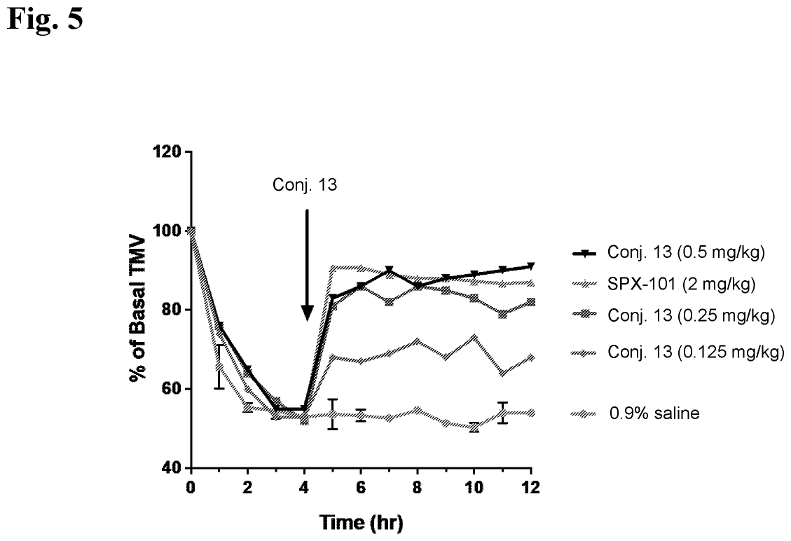

As shown in FIG. 5, the ENaC inhibitor Conj. 13 was tested at 0.125 mg/kg, 0.25 mg/kg, and 0.5 mg/kg and showed a dose-dependent restoration of TMV that was sustained over a period of 8 hours when compared to 0.9% saline control. A lower dose of Conj. 13 was needed to restore TMV (0.25 mg/kg) compared to SPX-101 (2 mg/kg), suggesting that Conj. 13 performed approximately 8-fold better than SPX-101 at restoring TMV in vivo. The data from SPX-101 at 2 mg/kg was extracted from Scott et al. (referenced above).

### Example 6. Synthesis of Conjugates Via Click Chemistry

General Procedure: Azide- or alkyne-modified peptide (1 equiv, crude), alkyne- or azide-modified amiloride or benzamil (1 equiv), CuSO₄·5H₂O (1 equiv), and ascorbic acid (1 equiv) were weighted into a reaction flask. Approx. 30% acetonitrile (ACN)/water was added to provide the peptide concentration of about 30 mg/mL. 4-Methylmorpholine (˜3-4 equiv.) was added which resulted in solution color change from light amber to red-brown. The resulting mixture was stirred at ambient temperature and the reaction progress was monitored by LC/MS until complete conversion was observed (˜3 h). The reaction was quenched by adjustment of pH to ˜3 by addition of trifluoroacetic acid (TFA). The resulting solution was a) lyophilized and purified or b) diluted with water, filtered, and purified by HPLC.

Conjugates 4-6, 13, and 15 were prepared using TM4 (see Table 3, azide-modified amiloride) and alkyne-modified peptide according to this example.

Conjugate 14 was prepared using TM5 (see Table 3, azide-modified benzamil) and alkyne-modified peptide according to this example.

Conjugates 7-9 were prepared using TM2 (see Table 3, alkyne-modified amiloride) and azide-modified peptide according to this example.

### Example 7. Synthesis of Conjugates Via Thiol-Maleimide Conjugation

General Procedure: A crude peptide with a side chain-deprotected Cys residue (1 equiv) was dissolved in ˜30-50% ACN/0.1 M ammonium acetate pH˜7.5 buffer at approx. 25 mg/mL. Separately, amiloride maleimide (˜1.25-1.5 equiv) was dissolved in ˜30-50% ACN/0.1 M ammonium acetate pH˜7.5 buffer at approx. 25 mg/mL. The two solutions were then mixed and stirred for a minimum of 1 h. The reaction progress was monitored by LC/MS until complete consumption of one of the starting material was observed. The reaction mixture was acidified to a target pH of ˜3 by addition of 10% aqueous TFA. The resulting solution was diluted (˜4×) with water then purified.

Conjugates 10-12 were prepared using TM6 according to this example.

### Example 8. Synthesis of Conjugates via Isothiourea Coupling (Amine-Coupling)

General Procedure: A peptide on resin with a deprotected Lys side chain (˜1 equiv) was suspended in dimethylformamide (DMF) (10 mL/g resin) to swell the resin. Amiloride isothiourea (1 equiv) was dissolved in DMF (5 mL/g resin). N-methylmorpholine (NMM) (˜3-4 equiv) was added to the amiloride isothiourea solution. The peptide-resin slurry was drained and the amiloride isothiourea/NMM/DMF solution was added to the resin. The resulting resin slurry was heated at 65-70° C. while stirring for a minimum of 12 h. A small sample of the resin was withdrawn for test cleavage and washed with DMF (6×), MeOH (2×), and methyl t-butyl ether (3×), then dried under vacuum for minimum of 2 h. The resin sample was subjected to a test cleavage using the cocktail composition of 93% TFA/2% H₂O/5% TIPS for ˜2.5-3.0 h. The obtained crude peptide conjugate was analyzed by LC/MS to confirm completion of the conjugation. Once the reaction was determined to be complete, the reaction mixture was allowed to cool down to ambient temperature. The resin was then washed with DMF (6-7×), IPA (2-3×), and MTBE (3×). The peptide resin was dried under vacuum for a minimum of 12 h then subjected to TFA cleavage.

Conjugates 1-3 were prepared using TM1 according to this example.

All of the compounds, compositions, and/or methods disclosed and claimed herein can be made and executed without undue experimentation in light of the present disclosure. While the compounds, compositions, and methods of this disclosure have been described in terms of preferred embodiments, it will be apparent to those of skill in the art that variations may be applied to the compounds, compositions, and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A conjugate of formula (I): 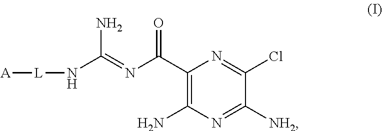
or a pharmaceutically acceptable salt thereof, wherein:
L is a bond, 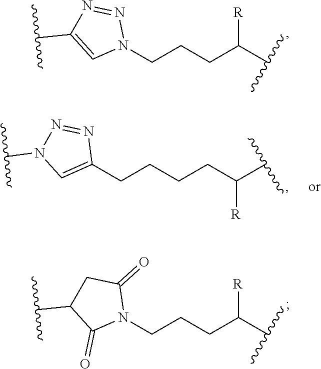
R is H or phenyl; and
A is a peptide designed to be retained in the lungs or a derivative thereof.

2. The conjugate of claim 1, wherein the peptide comprises ASHLRKLRKRL (SEQ ID NO: 1) or a derivative thereof.

3. The conjugate of claim 2, wherein the peptide derivative is acetylated, lipidated, amidated, derivatized with D-alanine, or derivatized with alpha-aminoisobutyric acid.

4. The conjugate of claim 1, wherein the peptide comprises SHLRKLRKRLL (SEQ ID NO: 58).

5. The conjugate of claim 1, wherein the peptide comprises ASHLRKLRKRLL (SEQ ID NO: 59).

6. The conjugate of claim 2, wherein the peptide comprises an additional leucine on the C-terminus of SEQ ID NO: 1.

7. The conjugate of claim 1, wherein the peptide is any one of SEQ ID NOs: 2-13 and 56-57.

8. The conjugate of any one of claims 1-7, wherein the peptide is bound to L at the C-terminus.

9. The conjugate of any one of claims 1-7, wherein the peptide is bound to L at the N-terminus.

10. The conjugate of any one of claims 1-7, wherein the peptide is bound to L at an amino acid side chain residue of the peptide.

11. The conjugate of claim 1, selected from any one of SEQ ID NOs: 14-55.

12. A pharmaceutical composition comprising any one of the conjugate of claims 1-11 and a pharmaceutically acceptable carrier or excipient.

13. A method of treating or preventing a disease or disorder in a subject in need thereof, comprising administering to the subject a therapeutically effective amount of the conjugate of any one of claims 1-11 or a pharmaceutical composition of claim 12.

14. The method of claim 13, wherein the disease or disorder is a pulmonary disease or disorder.

15. The method of claim 13, wherein the pulmonary disease or disorder is cystic fibrosis, idiopathic pulmonary fibrosis, chronic obstructive pulmonary disease, asthma, emphysema, primary ciliary dyskinesia, pneumonia, or non-cystic fibrosis bronchiectasis.

16. The method of any one of claims 13-15, wherein the subject is a mammal.

17. The method of any one of claims 13-16, wherein the subject is a human.

18. The method of any one of claims 13-17, wherein the conjugate or the pharmaceutical composition is administered once a day.

19. The method of any one of claims 13-18, wherein the conjugate or the pharmaceutical composition is administered multiple times a day.

20. The method of any one of claims 13-19, wherein the conjugate is administered at a dose ranging from about 0.1 mg/kg to about 100 mg/kg.

21. The method of any one of claims 13-20, wherein the conjugate or the pharmaceutical composition is administered intranasally, intratracheally, intrapulmonary, intrabronchially, or by inhalation.
